(19) United States
(12) Patent Application Publication
(10) Pub. No.: US 2026/0294471 A1
(43) Pub. Date: Oct. 1, 2026

(54) GUIDES AND TOOLS FOR MULTI-PORTAL SPINE PROCEDURES, AND ASSOCIATED SYSTEMS AND METHODS

(71) Applicant: Amplify Surgical, Inc., Foothill Ranch, CA (US)

(72) Inventors: Andy Wonyong Choi, Irvine, CA (US); Clark Hutton, Carlsbad, CA (US); Shigeru Tanaka, Half Moon Bay, CA (US); Martin Leugers, San Francisco, CA (US); Timothy Milberger, Sacramento, CA (US); Don Park, Irvine, CA (US)

(21) Appl. No.: 19/631,849

(22) Filed: Mar. 27, 2026

Related U.S. Application Data
(60) Provisional application No. 63/779,801, filed on Mar. 28, 2025.
(60) Provisional application No. 63/779,816, filed on Mar. 28, 2025.
(60) Provisional application No. 63/779,828, filed on Mar. 28, 2025.
(60) Provisional application No. 63/779,835, filed on Mar. 28, 2025.

(51) Int. Cl. A61B 17/34; A61B 17/32; A61B 17/3211

(52) U.S. Cl. CPC A61B 17/3403; A61B 17/32; A61B 17/3211; A61B 17/3494; A61B 2017/320052; A61B 2017/3407

(57) ABSTRACT

Guides for multi-portal spine procedures and associated systems and methods are disclosed herein. In some embodiments, a spinal surgical system for use in a spine procedure on a patient includes a spinal needle, a needle depth gauge, an angle plate guide, a scope port scalpel, a working port scalpel, a tissue dissector, and a flag guide. In some embodiments, the spinal surgical system can include (i) a plurality of angle plate guides including the angle plate guide and/or (ii) a plurality of flag guides including the flag guide. Different ones of the plurality of angle plate guides can have different sizes, and different ones of the plurality of flag guides can have different sizes.

### CROSS-REFERENCE TO RELATED APPLICATIONS

The present application claims the benefit of U.S. Provisional Patent Application No. 63/779,801, filed Mar. 28, 2025, U.S. Provisional Patent Application No. 63/779,816, filed Mar. 28, 2025, U.S. Provisional Patent Application No. 63/779,828, filed Mar. 28, 2025, and U.S. Provisional Patent Application No. 63/779,835, filed Mar. 28, 2025, the disclosures of which are incorporated herein by reference in their entireties.

### TECHNICAL FIELD

The present technology generally relates to medical systems and, more particularly, to guides and tools for multi-portal spine procedures, and associated systems and methods.

### BACKGROUND

Individuals often suffer from damaged or displaced spinal discs and/or vertebral bodies due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An intervertebral implant (commonly referred to as an interbody spacer or cage) can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is filled with bone and/or bone growth-inducing materials.

Unfortunately, it may be difficult to properly form and maintain access paths to a working space within the patient for the insertion of endoscopes, implant delivery instruments, and/or other surgical instruments. For example, a physician may attempt to form such access paths by trial-and-error and by taking multiple images (e.g., X-rays), causing a significant amount of trauma to the patient and requiring an extended surgery duration, leading to significantly increased recovery time and/or patient discomfort. Accordingly, there is a need for improved surgical systems and/or related technologies for guiding spine procedures.

### BRIEF DESCRIPTION OF THE DRAWINGS

FIG. 1 is an isometric view of a spinal surgical system configured in accordance with various embodiments of the present technology.

FIGS. 2A and 2B are schematic top plan and isometric views, respectively, showing surgical approaches to a lumbar spine for performing procedures.

FIG. 3 is an isometric view of a needle guide configured in accordance with various embodiments of the present technology.

FIG. 4 is a partially exploded, isometric view of the needle guide of FIG. 3.

FIG. 5 is an isometric view of a spinal needle configured in accordance with various embodiments of the present technology.

FIG. 6 is an isometric view of a needle depth gauge configured in accordance with various embodiments of the present technology.

FIGS. 7A and 7B are left isometric views of an angle plate guide in a closed state and in an open state, respectively, and configured in accordance with various embodiments of the present technology.

FIG. 8 is a partially exploded, left isometric view of the angle plate guide of FIG. 7A.

FIG. 9 is a right isometric view of the angle plate guide of FIG. 7A assembled with a guide rod in accordance with various embodiments of the present technology.

FIGS. 10A and 10B are isometric and proximal views, respectively, of a scope port scalpel configured in accordance with various embodiments of the present technology.

FIGS. 11A and 11B are isometric and proximal views, respectively, of a working port scalpel configured in accordance with various embodiments of the present technology.

FIG. 11C is an isometric view of working port scalpels of various sizes and each configured in accordance with various embodiments of the present technology.

FIGS. 12A and 12B are isometric and bottom views, respectively, of a tissue dissector configured in accordance with various embodiments of the present technology.

FIGS. 13A and 13B are isometric and top views, respectively, of a flag guide configured in accordance with various embodiments of the present technology.

FIG. 14 is a plan view of a surgical kit configured in accordance with various embodiments of the present technology.

FIGS. 15 and 16 illustrate operation of the needle guide of FIG. 3 in accordance with various embodiments of the present technology.

FIG. 17 illustrates operation of the spinal needle of FIG. 5 and the needle depth gauge of FIG. 4 in accordance with various embodiments of the present technology.

FIG. 18 illustrates operation of the angle plate guide of FIG. 7A, the scope port scalpel of FIG. 10A, and the working port scalpel of FIG. 11A in accordance with various embodiments of the present technology.

FIG. 19 illustrates operation of the angle plate guide of FIG. 7A and the tissue dissector of FIG. 10A in accordance with various embodiments of the present technology.

FIG. 20 illustrates operation of the flag guide of FIG. 13A in accordance with various embodiments of the present technology.

FIG. 21 is a flowchart illustrating a method for performing a multi-portal spine procedure in accordance with various embodiments of the present technology.

FIG. 22 is a side-view of a bone wax applicator positioned along a spine of a patient in accordance with at least some embodiments of the present technology.

FIG. 23 is an isometric view of a bone wax applicator in accordance with embodiments of the present technology.

FIG. 24 is an isometric view of the bone wax applicator of FIG. 23 in a loading configuration.

FIG. 25 is a top-plan view of the bone wax applicator of FIG. 23.

FIG. 26 is a cross-sectional view of the bone wax applicator taken along line 26-26 of FIG. 25.

FIG. 27 is a cross-sectional view of the bone wax applicator taken along line 26-26 of FIG. 25 in the loading configuration.

FIG. 28 is a top-back isometric view of an endoscope holder positioned along a back of patient in accordance with embodiments of the present technology.

FIG. 29 is a bottom-front isometric view of the endoscope holder of FIG. 28 in a first position in accordance with embodiments of the present technology.

FIG. 30 is a bottom view of the endoscope holder of FIG. 28 in accordance with embodiments of the present technology.

FIG. 31 is a top view of the endoscope holder of FIG. 28 in accordance with embodiments of the present technology.

FIGS. 32A and 32B are side views of the endoscope holder of FIG. 28 positioned along a patient in accordance with embodiments of the present technology.

FIG. 33 is a front view of the endoscope holder of FIG. 28 resting on a patient in accordance with embodiments of the present technology.

FIG. 34 is a back view of the endoscope holder of FIG. 28 resting on a patient in accordance with embodiments of the present technology.

FIG. 35 is an isometric view of a portion of the endoscope holder of FIG. 28 in a second position in accordance with embodiments of the present technology.

FIGS. 36A and 36B are side views of a spinal surgical system in an undeployed configuration and a deployed configuration, respectively, in accordance with at least some embodiments of the present technology.

FIG. 37 is a schematic cross-sectional side view of a dilator system of the spinal surgical system of FIG. 36A in accordance with at least some embodiments of the present technology.

FIG. 38 is an enlarged isometric view of the dissector device of the spinal surgical system of FIG. 36A in an insertion configuration in accordance with at least some embodiments of the present technology.

FIG. 39 is an enlarged isometric view of the dissector device of the spinal surgical system of FIG. 36A in accordance with at least some embodiments of the present technology.

FIGS. 40A, 40B, and 40C are enlarged, isometric views of the dissector device of the spinal surgical system of FIG. 36A being deployed in accordance with at least some embodiments of the present technology.

FIG. 41 is a flowchart illustrating a method for dissecting the fascial layer around a spine during a surgical procedure in accordance with at least some embodiments of the present technology.

### DETAILED DESCRIPTION

### I. Overview

The present technology is directed to instrument guides, bone wax applicators, endoscope holders, and balloon dissectors for multi-portal spine procedures. Many existing techniques for spine procedures, such as forming incisions, cutting through tissue to a desired depth and at an appropriate angle, delivering a spinal implant, and/or the like involve imaging (e.g., X-ray imaging), guesswork, and trial-and-error. For example, a physician may attempt to access a spinal disc based on tactile feedback and limited endoscope vision. Such “blind” triangulation can result in trauma and discomfort to the patient, extended surgery durations, and more. As discussed further herein, embodiments of the present technology can guide a physician in safely and accurately forming access paths to a working space of a patient, ensuring proper positioning of surgical instruments, and/or performing other aspects of spine procedures.

Some embodiments of the present technology are directed to guides for multi-portal spine procedures and associated systems and methods. The systems, devices, and methods disclosed herein can be used to create and maintain proper access paths to a working space in a patient. In particular, the access paths created can be tailored to the patient The access paths can be used for delivering irrigation fluid to the working space, removing a spinal disc, delivering a spinal implant (e.g., an expandable interbody device), and/or the like.

In some embodiments, a spinal surgical system for use in a spine procedure on a patient includes a spinal needle, a needle depth gauge, an angle plate guide, a scope port scalpel, a working port scalpel, a tissue dissector, and a flag guide. In some embodiments, the spinal surgical system can include (i) a plurality of angle plate guides including the angle plate guide and/or (ii) a plurality of flag guides including the flag guide. The angle plate guides can have different sizes, and the flag guides can have different sizes. A user can select appropriate guides (e.g., plate guides, flag guides, etc.) for the surgical procedure, surgical steps, etc.

In some embodiments, a method for performing a multi-portal spine procedure on a patient includes inserting a spinal needle into the patient until the spinal needle contacts a spine of the patient, moving a needle depth gauge relative to the spinal needle to obtain an indication of an insertion depth of the spinal needle, selecting an angle plate guide among a plurality of angle plate guides based on the obtained indication, and securing the selected angle plate guide to the patient and around the spinal needle.

Some embodiments of the present technology are directed to bone wax applicators. The surgical technology disclosed herein can be used to treat patients with, for example, stenosis, damaged or displaced spinal discs and/or vertebral bodies. During surgical procedure, injured tissue (e.g. anatomical elements, bones, etc.) can bleed, resulting in impaired viewing, blood loss, etc. A bone wax applicator can be used to apply bone wax to, for example, inhibit, limit, or substantially prevent internal bleeding to enhance visualization, reduce blood loss, or the like. The bone wax applicator can be used in endoscopic procedures, open procedures, or other procedures in which the surgical site may be accessed using ports, cannulas, or endoscopic techniques.

In some procedures, a bone wax applicator can apply bone wax before, during, and/or after implanting an implant. For example, instruments can be used to remove tissue along the spine to access an implantation site, prepare the implantation site, etc. The implant can then be inserted into, for example, an intervertebral implantation site between vertebral bodies to, for example, restore stability of the spine, provide lordotic correction, combinations thereof, or the like. In spinal fusion procedures, interbody devices can be used alone or in combination with bone, bone-growth-inducing materials, fixation devices (e.g., pedicle screw systems, fixation rods, etc.), or the like.

Visualization can help a surgeon throughout the surgical procedure to improve patient outcomes. Endoscopic visualization can be used to identify bleeding injury sites. Under endoscopic visualization, the bone wax applicator can be used to apply bone wax to injured tissue to inhibit, limit, or substantially such bleeding. For example, a site (e.g., decompression site, implantation site, etc.) and nearby tissue can be visualized using endoscopic techniques to view, for example, the spine (e.g., vertebral spacing, vertebral alignment, etc.), tissue (e.g., stenosis, damaged or displaced sections of intervertebral cartilage disc, tissue contributing to nerve compression, etc.), instruments and implants before, during, and after implantation, or the like.

In some embodiments, a bone wax applicator has different configurations for performing surgical steps. In a loading configuration, the bone wax applicator is configured for receiving and holding bone wax. In a dispensing configuration, the bone wax applicator can controllably dispense bone wax. The bone wax applicator can include a driver assembly that can be actuated to drive the bone wax along an internal passage of an endoscopic dispensing assembly. The bone wax can exit an angled distal tip for access to lateral regions along the spine. The configuration of the distal tip can be selected based on the location of the bleeding and can be replaced with additional tips for treatment flexibility.

Some embodiments of the present technology are directed to imaging surgical systems, endoscope holders, and endoscopes. The surgical systems can be used to treat patients with damaged or displaced spinal discs and/or vertebral bodies. The surgical systems can be used to perform endoscopic decompression procedures, implant a fixed or expandable interbody device to space apart vertebral bodies, restore stability of the spine, provide lordotic correction, combinations thereof, or the like. In spinal fusion procedures, endoscopes can be used to view the working spaces inside of the patient when performing, for example, decompression procedures, implantation procedures, etc. For example, the patient's spine can be visualized using endoscopic techniques to view, for example, the spine (e.g., vertebral spacing, vertebral alignment, etc.), tissue (e.g., damaged or displaced sections of intervertebral cartilage disc, tissue contributing to nerve compression, etc.), instruments and implants before, during, and after implantation, or the like. The visualization can help a surgeon throughout the surgical procedure to improve patient outcomes.

Embodiments of the present technology relate to surgical endoscope holders and associated devices and methods. The systems can be used to securely and effectively hold endoscopes over an extended period. The systems can also be conveniently adjustable to adapt to different working space demands. For example, an endoscope holder can include a stabilizer foot and a top guard. The top guard can include a bent portion that bends over a user's hand to distribute the weight of the endoscope holder and/or an endoscope over the back of a user's hand. Further, the stabilizer foot can be semicircular to distribute the weight of the endoscope and/or the endoscope holder over an area of a patient's skin while the endoscope is being used. As such, the endoscope holder can help distribute the weight of the endoscope across larger areas, reducing the strain on the surgeon's arms and hands from holding the endoscope for extended periods.

In some embodiments, the endoscope holder can include a cam assembly. The stabilizer foot can define a semicircular recess sized to house a portion of an endoscope and a cam body of the cam assembly can define a slot sized to house another portion of the endoscope. The endoscope can be placed in the semicircular recess and the slot to ensure that the endoscope is limited to, at a maximum, one-directional movement while held by the endoscope holder. Further, the cam body can further define a first recess and a second recess with profiles that concentrically align with the semicircular recess when the cam assembly is rotated. As such, the cam assembly can be rotated via a trigger to lock the endoscope into the first and second recesses. Thus, a user can lock the endoscope into place (e.g., fully restrained) by rotating the cam assembly. This allows the user full control over the endoscope while allowing the user to have a comfortable grip.

In some embodiments, the endoscope holder includes a plurality of stabilizer legs removably coupled to a body of the endoscope holder. The plurality of stabilizer legs can extend past the body of the endoscope holder in generally the same direction as the stabilizer foot. While in use, the stabilizer legs allow the user to create a tripod with the endoscope holder to distribute the weight of the endoscope and stabilize the endoscope. In effect, this reduces the strain on the surgeon over time and allows the surgeon to make more precise movements, reducing accidental trauma to tissue surrounding the endoscopic port.

Embodiments of the present technology can simplify holding endoscopes and other pieces of surgical equipment during a surgical procedure. The endoscope holder can easily hold an endoscope so that a surgeon can operate the endoscope while maintaining a comfortable grip. The endoscope holder includes a plurality of stabilizer legs that can be moved according to the specific needs of a patient, procedure, and/or user, enhancing surgical adaptability, flexibility, and precision. The endoscope holder also includes blunt, smooth, and/or curved surfaces and edges, minimizing the risk of injury to, for example, non-targeted tissue and/or the patient and ensuring safety during insertion and manipulation of the endoscope or other surgical equipment. Overall, endoscope holders configured in accordance with embodiments of the present technology enable users to easily stabilize an endoscope during a surgical procedure and to maintain their hold on the endoscope for an extended period of time.

Some embodiments of the present technology are directed to surgical dissector devices and associated devices and methods. The systems can be used to safely and effectively dissect tissues within a working space during a surgical procedure. The systems can also be conveniently adjustable to adapt to different working space demands and tissue types. For example, a spinal surgical system can include a dissector device, a tube, a stopcock, and a syringe. The dissector device can include an expandable dissector and an inflator assembly. The expandable dissector is configured to be controllably expanded along a patient's spine to, for example, manipulate tissue, push apart anatomical elements, enlarge working spaces, or the like. The inflator assembly can be fluidically coupled to the expandable dissector and include a housing and a plunger. The housing and plunger can be coupled so that the depression of the plunger into the housing can evacuate the expandable dissector from the housing. For example, the dissector device can be positioned adjacent to an opening such that when the plunger is depressed the expandable dissector is pushed into the opening. Further, a syringe (e.g., pump, inflation assembly) can be fluidly coupled to the dissector device via a tube and a stopcock. When the syringe is actuated (e.g., depressed) the expandable dissector can be filled with air to dissect the tissue. The syringe can then be actuated again (e.g., withdrawn) to deflate the expandable dissector and the spinal surgical system can be removed from the patient.

In some embodiments, the stopcock can be used to reduce the bulk of the spinal surgical system while it is in use. For example, after the expandable dissector has been inflated, the stopcock can be closed so that the air in the expandable dissector is trapped inside a closed system. The syringe can then be removed from the system and placed to the side, reducing the overall size and weight of the system and enabling easier maneuvering of the dissector device inside of the patient. Further, the stopcock can then be opened to allow the expandable dissector to deflate without reattaching the syringe.

In some embodiments, dilators can be inserted into the patient via ports to create a working space for the dissection device. For example, a dilator system can include a first dilator and a second dilator that are inserted into the patient via two separate ports spaced apart from each other. The dilators can be inserted at different angles to create working spaces of varying widths around the fins of the spine. The first and second dilators can create a working channel that additional surgical instruments, such as the dissector device, can extend through to the working space.

In some embodiments, a dissector device can include a retractable cover tip configured to create an opening in soft tissue via one or more cutting edges. For example, a cutting edge of the cover tip can be scraped along a lamina to create an opening in the fascial layer. The cover tip can then be retracted to prevent additional damage to the surrounding tissues (e.g., non-target tissues) and optionally, to deploy an expandable dissector (e.g., a balloon, an anatomically shaped balloon, etc.) into the opening. In some embodiments, a housing of the dissector device can include locking mechanisms to prevent the retracted cover tip from expanding once it has been retracted.

In some embodiments, a dissector device can include a flange configured to support an inflated expandable dissector and to maintain the expandable dissector's position relative to the dissector device. For example, the expandable dissector can be expanded by depressing a fluidly coupled syringe to fill the expandable dissector with fluid or gas. The expandable dissector can be of a predetermined size and shape specific to an operation, patient, and/or user. The flange can support the expandable dissector as it expands and ensure that the expandable dissector does not move out of position when it is only partially inflated. In some aspects of the present technology, this lowers the risk of accidental trauma to tissues other than the target tissues (e.g., non-target tissues). In further embodiments, the cover tip can be excluded and the flange can act as an alternative cutting edge for scraping the lamina.

In some embodiments, a method of operating a spinal surgery system includes the steps of (i) inserting a first and a second dilator into a patient through a first and second port, respectively, (ii) dilating tissue of the patient to define a working space, (iii) inserting a dissector device into the working space, (iv) scraping a cover tip along the lamina of the spine to create an opening in the fascial layer, (v) retracting the cover tip and positioning a proximal portion of the dissector device at the opening, (vi) depressing a plunger to evacuate an expandable dissector from the dissector device, (vii) actuating a syringe (e.g., depressing a plunger) to inflate the expandable dissector and dissect the fascial layer, (viii) actuating the syringe (e.g., extending the plunger) to deflate the expandable dissector, and (ix) removing the dissector device and dilators from the patient.

In some embodiments, a method of operating a spinal surgery system includes the steps of (i) inserting at least one dilator into a patient through a port, (ii) dilating tissue of the patient to access a working space, (iii) inserting a dissector device into the working space, (iv) scraping a cover tip along the lamina of the spine to create an opening in the fascial layer, (v) retracting the cover tip and positioning a proximal portion of the dissector device at the opening, (vi) depressing a plunger to deploy an expandable dissector from the housing, (vii) depressing a plunger of the pump (e.g., syringe pump, manual pump, etc.) to inflate the expandable dissector and dissect, for example, the fascial layer, (viii) closing a stopcock and uncoupling the pump, (ix) reattaching the pump to the system after a period of time, (x) actuating the syringe (e.g., extending a plunger) to deflate the expandable dissector, and/or (xi) removing the dissector device from the patient.

In some embodiments, a method of operating a spinal surgery system includes the steps of (i) inserting one or more dilators into a patient through at least one port, (ii) dilating tissue of the patient, (iii) inserting a dissector device toward a target working space in a subject, (iv) scraping a cover tip along tissue to access a dissection location (e.g., lamina of the spine to create an opening in the fascial layer), (v) retracting the cover tip and positioning a proximal portion of the dissector device at the opening, (vi) depressing an applicator plunger to deploy an expandable dissector from the dissector device, (vii) depressing a plunger of a syringe to inflate the expandable dissector and dissect tissue, such as the fascial layer, push apart tissue, enlarge a working space, etc., (viii) closing a stopcock and uncoupling the syringe to hold the expandable dissector in an inflated configuration, (ix) opening the stopcock to deflate the ballon, and/or (x) removing the dissector device and dilators from the patient.

In some embodiments, the dissector system is used in a spinal surgical system that also includes a visualization instrument and/or other devices to assist the surgical procedure, including instrument holders, pillows, etc. Surgical techniques described herein can include a spinal implant delivery procedure, a decompression procedure, an oblique lumbar interbody fusion (OLIF) procedure, a lateral lumbar interbody fusion (LLIF) procedure, a posterior lumbar interbody fusion (PLIF) procedure, a transforaminal lumbar interbody fusion (TLIF) procedure, an anterior lumbar interbody fusion (ALIF) procedure, or combinations thereof.

Embodiments of the present technology can simplify dissecting tissues during a surgical procedure. The dissector device can easily create an opening in the fascial layer and then be inserted and actuated to dissect the surrounding tissue. The dissector device includes a versatile expandable dissector that can be manufactured according to the specific needs of a patient, procedure, and/or user, enhancing surgical adaptability, flexibility, and precision. The dissector device also includes blunt, smooth, and/or curved surfaces and edges, minimizing the risk of injury to, for example, non-targeted tissue and/or the patient and ensuring safety during insertion and manipulation. Overall, dissector devices configured in accordance with embodiments of the present technology enable users to safely and efficiently separate tissue from the bone to allow easy insertion of additional instruments and/or implants.

In the following description, specific details are set forth to provide a thorough understanding of aspects of the present technology. One skilled in the relevant art will recognize, however, that the systems, devices, and techniques described herein can be practiced without one or more of the specific details set forth herein, or with other methods, components, materials, etc.

Reference throughout this specification to an “example” or an “embodiment” means that a particular feature, structure, or characteristic described in connection with the example or embodiment is included in at least one example or embodiment of the present technology. Thus, use of the phrases “for example,” “as an example,” or “an embodiment” herein are not necessarily all referring to the same example or embodiment and are not necessarily limited to the specific example or embodiment discussed. Furthermore, features, structures, or characteristics of the present technology described herein may be combined in any suitable manner to provide further examples or embodiments of the present technology.

Spatially relative terms (e.g., “beneath,” “below,” “over,” “under,” “above,” “upper,” “top,” “bottom,” “left,” “right,” “center,” “middle,” and the like) may be used herein for ease of description to describe one element's or feature's relationship relative to one or more other elements or features as illustrated in the figures. It will be understood that the spatially relative terms are intended to encompass different orientations of a device or system in use or operation, in addition to the orientation depicted in the figures. For example, if a device or system illustrated in the figures is rotated, turned, or flipped about a horizontal axis, elements or features described as “below” or “beneath” or “under” one or more other elements or features may then be oriented “above” the one or more other elements or features. Thus, the exemplary terms “below” and “under” are non-limiting and can encompass both an orientation of above and below. The device or system may additionally, or alternatively, be otherwise oriented (e.g., rotated ninety degrees about a vertical axis, or at other orientations) than illustrated in the figures, and the spatially relative descriptors used herein are interpreted accordingly. In addition, it will also be understood that when an element is referred to as being “between” two other elements, it can be the only element between the two other elements, or one or more intervening elements may also be present.

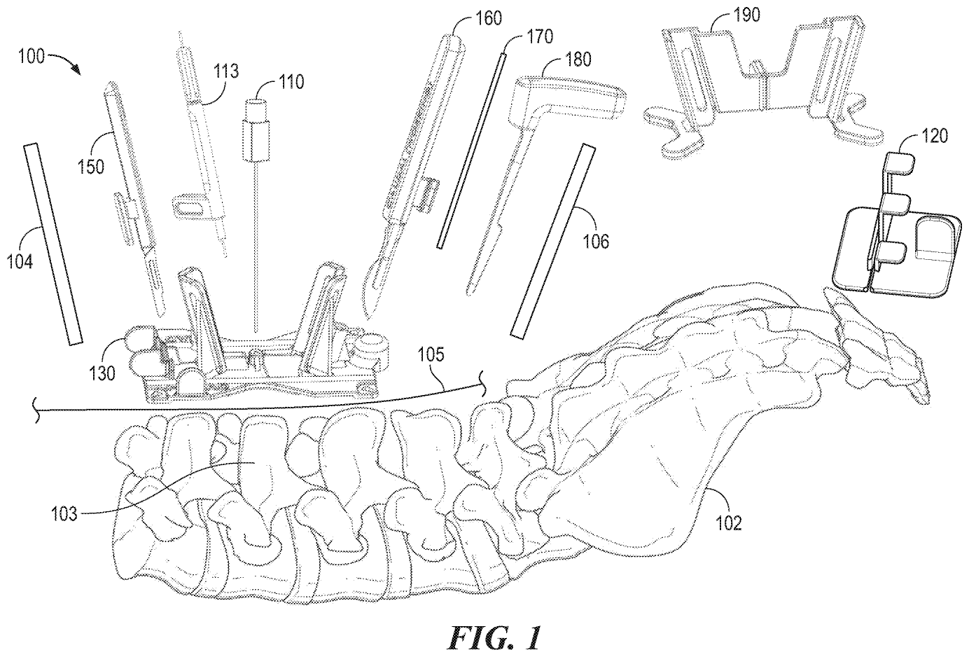

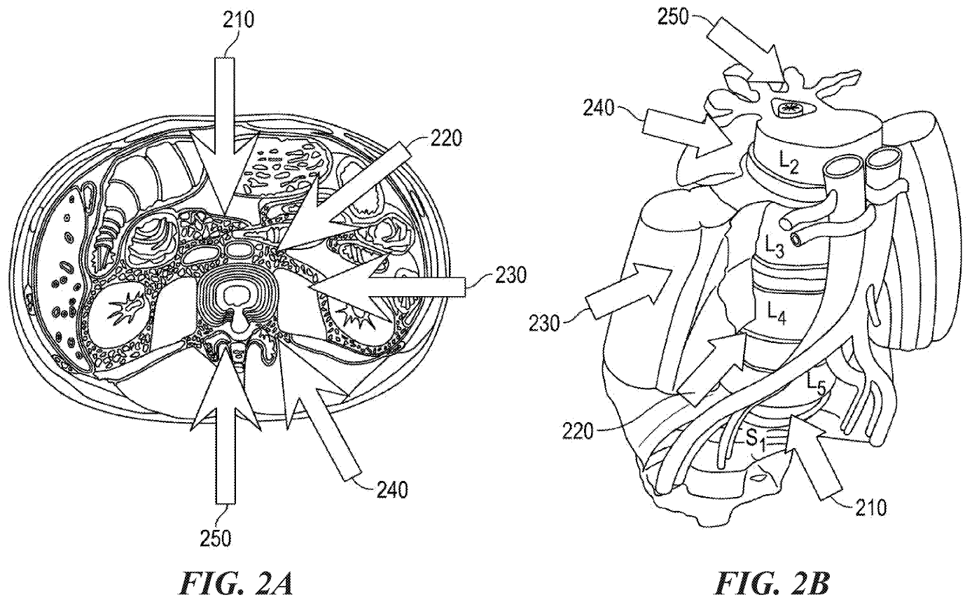

Reference numbers used in the figures of the present disclosure follow a numbering convention in which (i) the first digit or digits correspond to the first figure in which a particular element or component is introduced and (ii) the remaining digits identify that particular element or component in the figures. Unless otherwise specified or made clear from context, similar references numbers are used across multiple figures to denote generally similar and/or identical components. For example, reference number 102 can be used to reference an element “2” that was first introduced in FIG. 1. Use of reference number 102 in FIG. 2 can identify the element “2” from FIG. 1 in FIG. 2. Use of reference number 202 in FIG. 2 can be used to reference an element “2” that was first introduced in FIG. 2, and that may (depending on context) be generally similar and/or identical to the element “2” corresponding to reference number 102 that was first introduced in FIG. 1.

### II. Select Embodiments of Instrument Guides for Spinal Surgical Systems

FIG. 1 is an isometric view of a spinal surgical system 100 (“the system 100”) positioned adjacent to a human patient's spine 102 and configured in accordance with various embodiments of the present technology. The system 100 can include a set of instruments and guides for performing surgical steps. The guides can be used to position and/or orient the instruments. In some embodiments, the system 100 can include, for example, a needle guide 113, a spinal needle 110, a needle depth gauge 120, an angle plate guide 130, a scope port scalpel 150, a working port scalpel 160, a guide rod 170, and a tissue dissector 180. The system 100 can also include a visualization instrument 104, a working instrument 106, and a flag guide 190.

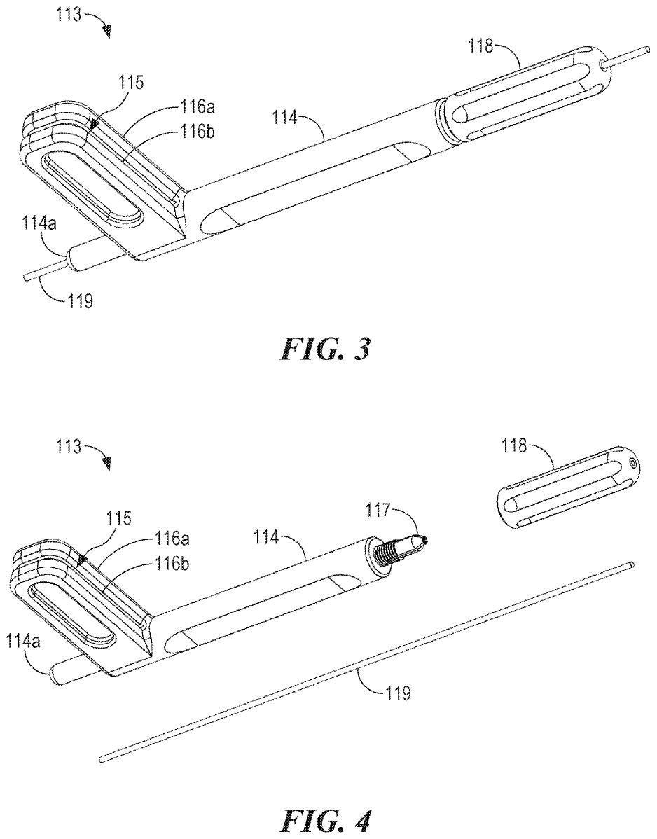

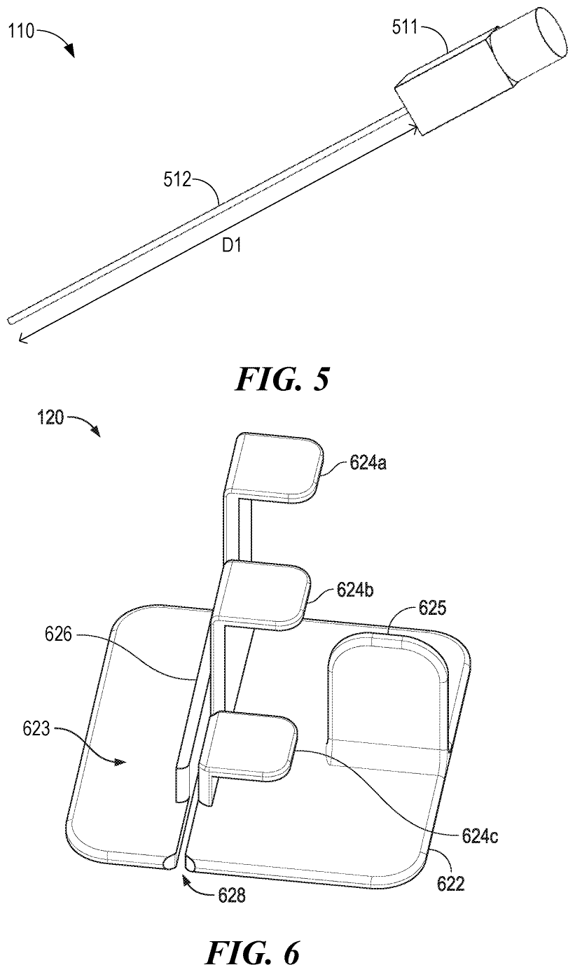

The spinal needle 110 can be shaped and sized to pierce the patient's skin 105 and reach, for example, a targeted depth, anatomical feature (e.g., a lamina 103 of the spine 102, intervertebral disc, etc.), interspinous space, etc. The needle guide 113 can be used to properly position the spinal needle 110 with respect to the patient's spine 102. The needle guide 113 is described in greater detail below with reference to FIGS. 3 and 4, and the spinal needle 110 is described in greater detail below with reference to FIG. 5. The needle depth gauge 120 can be positioned on the patient's skin 105 while the spinal needle 110 is inserted in the patient to measure an insertion depth of the spinal needle 110. The needle depth gauge 120 is described in greater detail below with reference to FIG. 6.

The angle plate guide 130 can be shaped and sized to guide one or more surgical tools into the patient. For example, the angle plate guide 130 can guide the scope port scalpel 150, the working port scalpel 160, and/or the tissue dissector 180 to appropriate positions and at appropriate angles to assist with forming two or more incisions on the patient's skin 105. The two or more incisions can define two or more ports for a subsequent multi-portal spine procedure. For example, the angle plate guide 130 can guide the scope port scalpel 150 in creating a first port (and corresponding first access path) for insertion of the visualization instrument 104 (e.g., an endoscope). As another example, the angle plate guide 130 can guide the working port scalpel 160 and/or the tissue dissector 180 in creating a second port (and corresponding second access path) spaced apart from the first port and for insertion of the working instrument 106 (e.g., an implant delivery instrument). In some embodiments, the guide rod 170 helps guide the working port scalpel 160 along the angle plate guide 130.

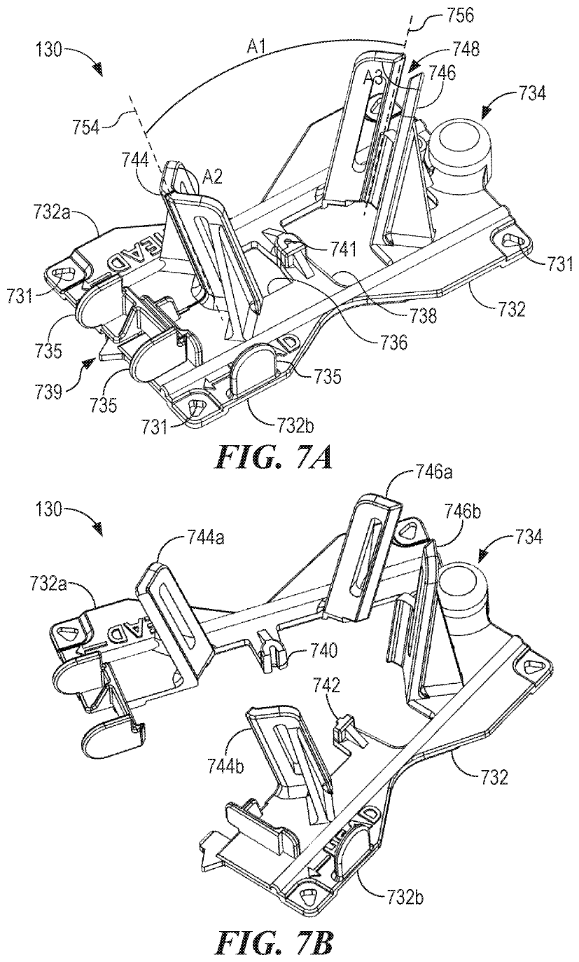

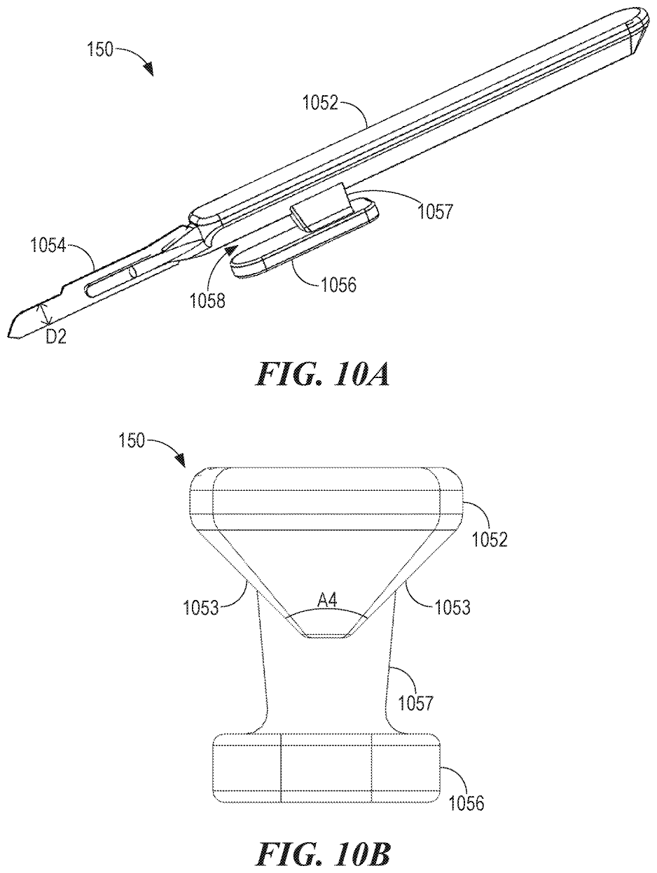

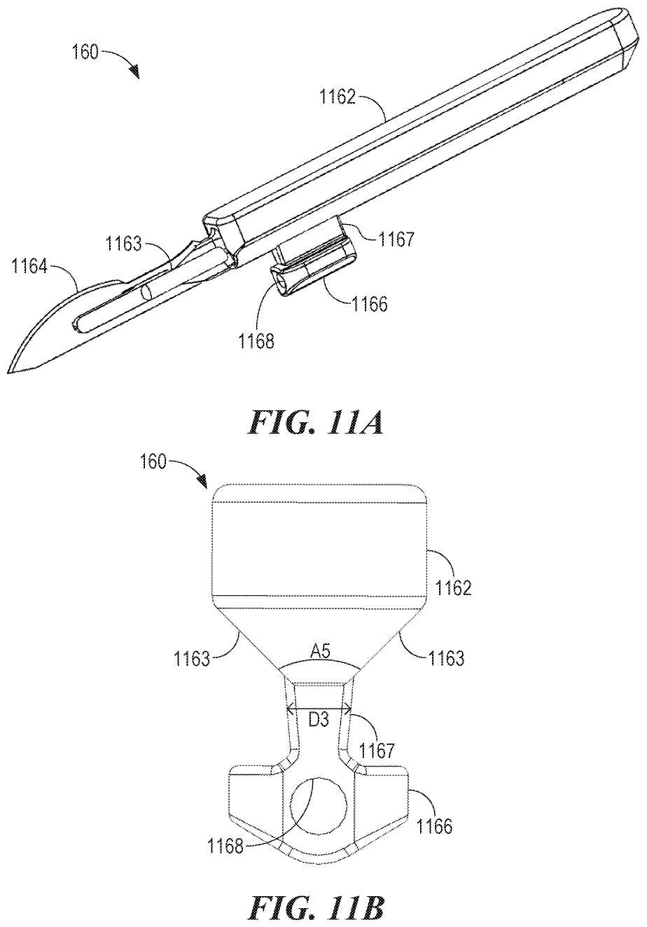

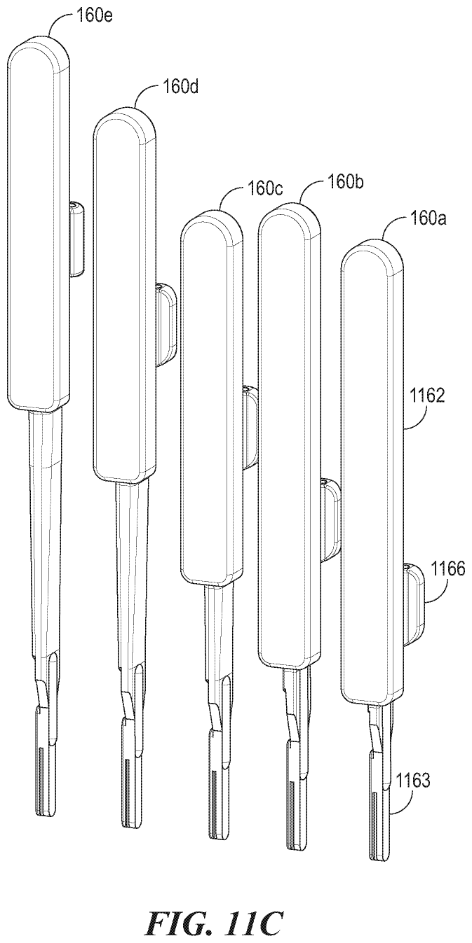

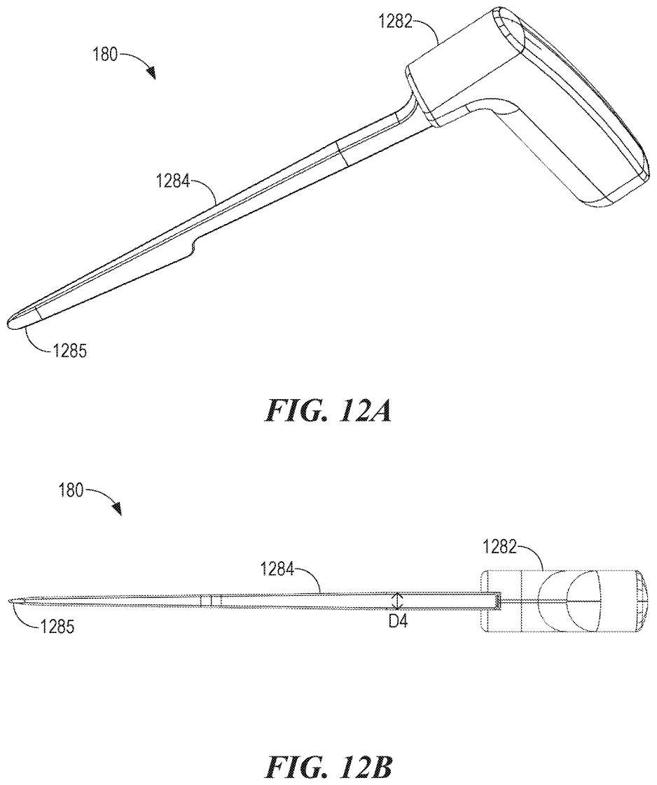

As discussed in further detail herein, a plurality of angle plate guides 130 having different sizes, angles, and/or other configurations may be available, and the measurements taken using the needle depth gauge 120 can inform the physician which of the plurality of angle plate guides 130 should be used for the particular patient. Thus, embodiments of the present technology enable the formation of patient-tailored ports and corresponding access paths to the working space in the patient. The angle plate guide 130 is described in greater detail below with reference to FIGS. 7A-7. The scope port scalpel 150 is described in greater detail below with reference to FIGS. 10A and 10B. The working port scalpel 160 is described in greater detail below with reference to FIGS. 11A and 11B. The tissue dissector 180 is described in greater detail below with reference to FIGS. 12A and 12B.

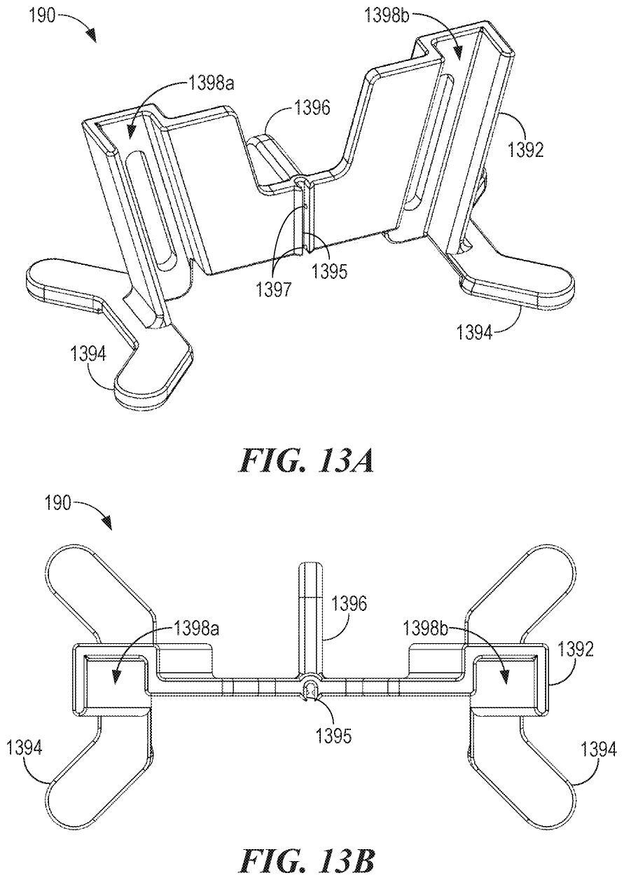

Once the ports are formed, the visualization instrument 104 and the working instrument 106 can each be inserted into the corresponding port and access path to visualize or operate at, respectively, the working space in the patient. In some embodiments, the flag guide 190 can be positioned on the patient's skin 105 to confirm and/or ensure that each of the visualization instrument 104 and the working instrument 106 is positioned and oriented properly. As discussed in further detail herein, a plurality of flag guides 190 having different sizes, angles, and/or other configurations may be available, and the measurements taken using the needle depth gauge 120 can inform the physician which of the plurality of flag guides 190 should be used for the particular patient. Thus, embodiments of the present technology enable the maintenance of patient-tailored ports and corresponding access paths to the working space in the patient. The flag guide 190 is described in greater detail below with reference to FIGS. 13A and 13B.

It is appreciated that the illustrated system 100 is merely a representative example, and that the system 100 can include additional, fewer, and/or alternative components. For example, the system 100 can additionally include an irrigation fluid delivery assembly (e.g., an irrigation fluid source and a pump). As another example, the system 100 can additionally include an ergonomic pillow or other component for supporting one or more body parts of the physician, such as their hand, wrist, or forearm.

FIGS. 2A and 2B are schematic top plan and isometric views, respectively, showing surgical approaches to a lumbar spine for performing procedures. The surgical approaches illustrated and described herein can be used for performing procedures suitable for the system 100 of FIG. 1 and/or other systems disclosed herein. Referring to FIGS. 2A and 2B together, surgical instruments (e.g., the working instrument 106) can be delivered via different paths, including an ALIF path 210, an OLIF path 220, an LLIF or extreme lateral lumbar interbody fusion (XLIF) path 230, a TLIF path 240, and a PLIF path 250. These paths can be used to deliver spinal implants to a target site on a patient's spine. These paths can also be used to perform other procedures disclosed herein. For example, one or more of the paths 210, 220, 230 240, 250 can be selected for multi-portal endoscopic approaches to perform a wider array of lumbar spine procedures than conventional one-portal techniques. Devices can be positioned along the same path or different paths to allow for independent positioning and manipulation of the endoscopic camera of the surgical instruments, thereby providing greater flexibility and enhanced visualization of spinal anatomy.

Surgical instruments can remove tissue to define working space(s) inside the patient. In one example TLIF procedure, the transforaminal path 240 may be employed to implant a single small expandable or non-expandable interbody spacer at the intervertebral space. In one example PLIF procedure, two interbody spacers can be delivered along the posterior path 250 and implanted at the intervertebral space. The two interbody spacers can cooperate to keep the vertebral bodies at the desired spacing and may be larger than the TLIF spacer. Additionally, multiple interbody spacers can provide lordotic correction by providing support at different heights. In one example LLIF procedure, a single, relatively large interbody spacer can be delivered along the lateral path 230 and implanted to provide asymmetrical support. In one example ALIF procedure, an asymmetric interbody spacer can be delivered along the anterior path 210 to provide support consistent with lordosis at that portion of the spine. Lateral approaches, transforaminal approaches, and anterior approaches can be used to access the cervical spine, thoracic spine, etc. The number of instruments, configurations of instruments, implants, and surgical techniques can be selected based on the condition to be treated.

FIG. 3 is an isometric view of the needle guide 113. The needle guide 113 can include a positioner 114, a tightening knob 118, and a wire or rod 119. The positioner 114 can have a generally elongate body with a distal end 114a, and a pair of wings 116a and 116b (collectively referred to as “the wings 116”) extending from the elongate body in a generally perpendicular direction. The wings 116 can define a narrow gap 115 therebetween, which can be sized to receive the spinal needle 110. The positioner 114 and the tightening knob 118 can co-define a channel sized to receive the rod 119 therethrough, as shown. The rod 119 can be radiopaque.

FIG. 4 is a partially exploded, isometric view of the needle guide 113. As shown, the positioner 114 can also include a clamp or chuck 117 (e.g., having three flexible, cantilevered members). To assemble the needle guide 113, a user can insert the rod 119 into the channel of the positioner 114 either from the distal end 114a or the chuck 117, position the rod 119 along the length of the positioner 114 as desired, then couple the tightening knob 118 to the chuck 117 (e.g., by engaging corresponding threads) until the tightening knob 118 tightens the chuck 117, which then grips onto the rod 119 in a secure manner.

FIG. 5 is an isometric view of the spinal needle 110. The spinal needle 110 can include a handle 511 (also referred to herein as “the needle hub 511”) and a needle 512 extending from the handle 511. The needle 512 can have a length D1 between 1-10 inches or between 5-4 inches, such as about 5.5 inches. In operation, the physician can grip the handle 511 and insert the needle 512 into the patient's back until, for example, tactile feedback indicates that the needle 512 has reached a portion of the patient's spine (e.g., the lamina 103).

FIG. 6 is an isometric view of the needle depth gauge 120 (also referred to herein as “the plate guide selector 120”). The needle depth gauge 120 can include a substantially flat base 622 and (i) a plurality of depth indicators (individually labeled 624a-624c, collectively referred to as “the depth indicators 624”), (ii) a protrusion 625, and (iii) a guiding wall 626 each extending upward from the base 622. The base 622 can include a slot 628 sized to receive the needle 512 of the spinal needle 110. The base 622 can also include markings 623 instructing the user (e.g., the physician) how to use the needle depth gauge 120. For example, in the illustrated embodiment, the markings 623 include the text “INSERT NEEDLE” and an arrow extending parallel to the slot 628. The protrusion 625 can serve as a handle for manipulating the needle depth gauge 120 and/or a support for the physician's finger. The needle depth gauge 120 can be composed of plastic, metal, and/or other suitable material, and can be manufactured via molding, additive manufacturing (e.g., 5D printing), machining, and/or other suitable techniques.

The depth indicators 624 can be shaped as L-brackets having different heights, with the tallest depth indicator 624a farthest from the opening of the slot 628, the shortest depth indicator 624c closest to the opening of the slot 628, and the intermediate depth indicator 624b therebetween. Moreover, as shown, the tallest depth indicator 624a can be marked with a “1,” the intermediate depth indicator 624b can be marked with a “2,” the shortest depth indicator 624c can be marked with a “3,” and the portion of the base 622 adjacent to the opening of the slot 628 can be marked with a “4.” Operation of the spinal needle 110 and the needle depth gauge 120 is described in further detail herein with reference to FIG. 13.

FIGS. 7A and 7B are left isometric views of the angle plate guide 130 in a closed state and in an open state, respectively. Referring first to FIG. 7A (closed state), the angle plate guide 130 can include a first base portion 732a and a second base portion 732b (collectively referred to as “the base 732” or “the articulatable base 732”), a pivot 734, a latch 739, a first guiding ramp 744, and a second guiding ramp 746. The base 732 can be substantially flat and can include one or more apertures 731 at the corners thereof. The one or more apertures 731 can be sized to receive needles, staples, sutures, and/or the like for releasably securing the base 732 (and thus the angle plate guide 130) to the patient's skin 105. The base 732 can also include one or more protrusions 735 (FIG. 7A illustrates three protrusions 735) that can serve as handles for manipulating the angle plate guide 130 and/or supports for the physician's fingers. When the angle plate guide 130 is in the closed state, the base 732 can also define (i) a first opening 736, (ii) a second opening 738, and (iii) an aperture 741. The first opening 736 and the second opening 738 can be shaped and sized to receive the visualization instrument 104 and the working instrument 106 (FIG. 1) respectively. The aperture 741 can be shaped and sized to receive the needle 512 of the spinal needle 110. The angle plate guide 130 can be composed of plastic, metal, and/or other suitable material, and can be manufactured via molding, additive manufacturing (e.g., 5D printing), machining, and/or other suitable techniques.

Each of the first guiding ramp 744 and the second guiding ramp 746 can extend upward from the base 732 at an angle. In the illustrated embodiment, the first guiding ramp 744 and the second guiding ramp 746 form an angle A1 therebetween. As discussed in further detail herein, the angle A1 and/or the distance between the first guiding ramp 744 and the second guiding ramp 746 can vary such that the first guiding ramp 744 and the second guiding ramp 746 extend along axes 754, 756, respectively, that generally intersect at or near the working space. The axes 754, 756 can intersect at a location within the target working space to, for example, avoid contacting deeper non-targeted tissue (e.g., nerve tissue, spinal cord, etc.) with the tools or instruments. For example, the angle A1 can be between 12-170 degrees or between 20-40 degrees, such as about 50 degrees. Also, each of the first guiding ramp 744 and the second guiding ramp 746 can have a generally V-shaped cross-section. The angle A1 can be increased or decreased to decrease or increase, respectively, the targeted depth for the instruments or tools. The guiding ramp 744 and the second guiding ramp 746 can have fixed positions or can be adjustable. In adjustment embodiments, the guiding ramp 744 and second guiding ramp 746 can be pivotally coupled (e.g., via lockable hinges or pivots) to the articulatable base 732.

In the illustrated embodiment, the first guiding ramp 744 has a continuous V-shaped cross-section with an angle A2, and the second guiding ramp 746 has a discontinuous V-shaped cross-section defining a gap 748 and with an angle A3. Each of the angle A2 and the angle A3 can be between 90-110 degrees, such as about 110 degrees. In other embodiments, however, the guide ramps 744, 746 can have other shapes, such as a tube, a lumen, a flat surface, a rounded (e.g., concave) surface, etc. Accordingly, the term “guiding ramp” as used herein shall not be limited to specific geometries, but inclusive of structures that can guide various instruments toward a target site, as discussed in greater detail herein.

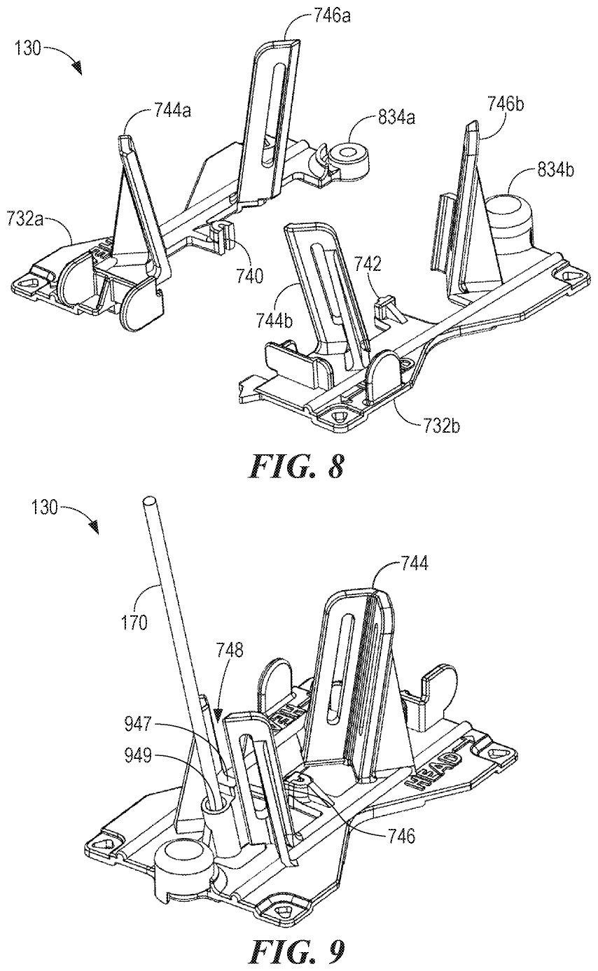

The pivot 734 and the latch 739 can be used to transition the angle plate guide 130 between the closed state and the open state. Referring momentarily to FIG. 8, which is a partially exploded, left isometric view of the angle plate guide 130, the first base portion 732a can include a first rotational portion 834a and the second base portion 732b can include a second rotational portion 834b corresponding to the first rotational portion 834a. The second rotational portion 834b can rotate relative to the first rotational portion 834a via a bearing, a screw, a rod, and/or the like. Referring next to FIG. 7B (open state), the first base portion 732a and the second base portion 732b can be rotated relative to one another about the pivot 734. As shown, the first base portion 732a can include a first ramp portion 744a and a second ramp portion 746a, and the second base portion 732b can include a third ramp portion 744b and a fourth ramp portion 746b. When the angle plate guide 130 is transitioned to the closed state (FIG. 7A), the first ramp portion 744a and the third ramp portion 744b can form the first guiding ramp 744, and the second ramp portion 746a and the fourth ramp portion 746b can form the second guiding ramp 746.

As shown, the first base portion 732a can also include a recess or open channel 740, and the second base portion 732b can also include an elongate protrusion 742. The open channel 740 can be shaped and sized to receive the needle 512 of the spinal needle 110 (FIG. 5) and the elongate protrusion 742. Thus, in operation, while the spinal needle 110 is inserted into the patient and extending above the patient's skin 105, the physician can position the angle plate guide 130 in the open state (FIG. 7B) around the spinal needle 110. Subsequently, the physician can rotate the first base portion 732a and/or the second base portion 732b about the pivot 734 until (i) the open channel 740 and the elongate protrusion 742 form the aperture 741 and enclose the needle 512 therebetween and (ii) the latch 739 (e.g., via a snap-fit mechanism, etc.) secures the angle plate guide 130 in the closed state (FIG. 7A). The physician may then optionally secure the base 732 to the patient's skin via the one or more apertures 731. Accordingly, the position and/or the orientation of the angle plate guide 130 can be secured relative to the spinal needle 110.

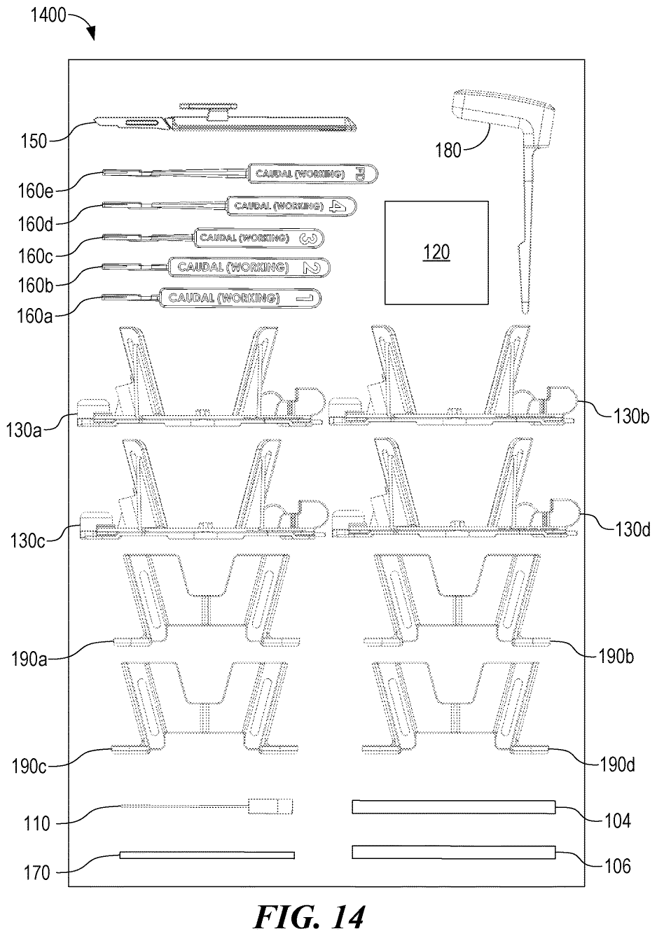

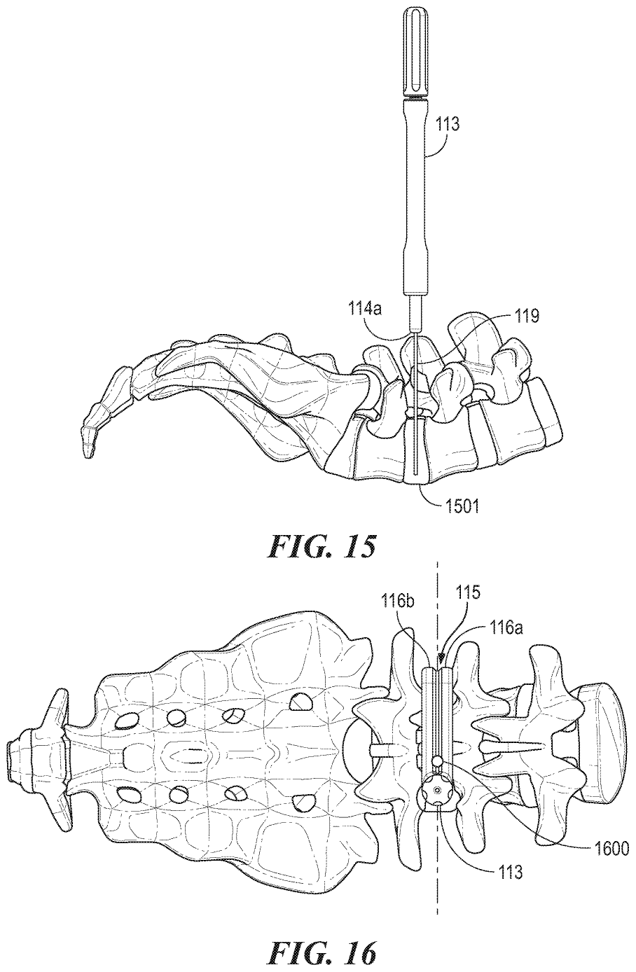

FIG. 9 is a right isometric view of the angle plate guide 130 assembled with the guide rod 170 in accordance with various embodiments of the present technology. As shown, the second guiding ramp 746 can include (i) a stop 947 (e.g., a surface extending between the second ramp portion 746a and the fourth ramp portion 746b) below the gap 748 and (ii) an aperture or recess 949 shaped and sized to receive the guide rod 170 therein. As discussed further herein with reference to FIGS. 14 and 15, the guide rod 170, the stop 947, and the gap 748 can facilitate use of the working port scalpel 160 and the tissue dissector 180 (among other instruments) with the angle plate guide 130.

FIGS. 10A and 10B are isometric and proximal views, respectively, of the scope port scalpel 150. Referring first to FIG. 10A, the scope port scalpel 150 can include a handle 1052, a blade 1054 extending from the handle 1052, a connector 1057, and a clip 1056 coupled to the handle 1052 via the connector 1057. The blade 1054 can have a width D2 between 5-7 mm, such as about 7 mm. The handle 1052 and the clip 1056 can define a gap 1058 therebetween sized to receive the first guiding ramp 744 of the angle plate guide 130 (FIG. 7A). In particular, as discussed in further detail herein with reference to FIG. 14, the position of the connector 1057 along the length of the handle 1052 can define a maximum insertion depth of the scope port scalpel 150.

Referring next to FIG. 10B, the handle 1052 can have a pair of bottom surfaces 1053 that generally form a V-shape and define an angle A4 (e.g., 110 degrees). In particular, the angle A4 can be identical or generally similar to the angle A2 of the first guiding ramp 744 (FIG. 7A). Therefore, as discussed in further detail herein with reference to FIG. 14, the handle 1052 can be positioned generally flush against the first guiding ramp 744 and the corresponding V-shapes can reduce lateral movement of the scope port scalpel 150 relative to the angle plate guide 130.

FIGS. 11A and 11B are isometric and proximal views, respectively, of the working port scalpel 160. Referring first to FIG. 11A, the working port scalpel 160 can include a handle 1162, a blade holder 1163 extending from the handle 1162, a blade 1164 coupled to or otherwise held by the blade holder 1163, a connector 1167, and a sleeve 1166 coupled to the handle 1162 via the connector 1167. The sleeve 1166 can define a channel 1168 shaped and sized to receive the guide rod 170 (FIG. 9) therethrough. Thus, the guide rod 170 and the sleeve 1166 can help guide the working port scalpel 160 along the second guiding ramp 746 (FIG. 7A). Also, as discussed in further detail herein with reference to FIG. 14, the position of the connector 1167 along the length of the handle 1162 can define a maximum insertion depth of the working port scalpel 160.

Referring next to FIG. 11B, the handle 1162 can have a pair of bottom surfaces 1163 that generally form a V-shape and define an angle A5 (e.g., 110 degrees). In particular, the angle A5 can be identical or generally similar to the angle A3 of the second guiding ramp 746 (FIG. 7A). Also, the connector 1167 can have a width D3 less than the gap 748 (FIG. 7A) so that the connector 1167 can fit therein. Therefore, as discussed in further detail herein with reference to FIG. 14, the handle 1162 can be positioned generally flush against the second guiding ramp 746 and the corresponding V-shapes can reduce lateral movement of the working port scalpel 160 relative to the angle plate guide 130.

FIG. 11C is an isometric view of working port scalpels 160a-160e of various sizes and each configured in accordance with various embodiments of the present technology. Corresponding blades (e.g., the blade 1164 of FIG. 11A) of the working port scalpels 160a-160e are omitted for illustrative purpose only. As shown, the working port scalpels 160a-160e can have different sizes (e.g., lengths) of their handle 1162 and/or blade holder 1163. In the illustrated embodiment, for example, the working port scalpel 160e has a longer blade holder 1163 than the working port scalpel 160a, and the working port scalpel 160a has a longer handle 1162 than the working port scalpel 160c. Moreover, the sleeves 1166 of the working port scalpels 160a-160e can be positioned at different distances from the distal tip of the corresponding blade holders 1163.

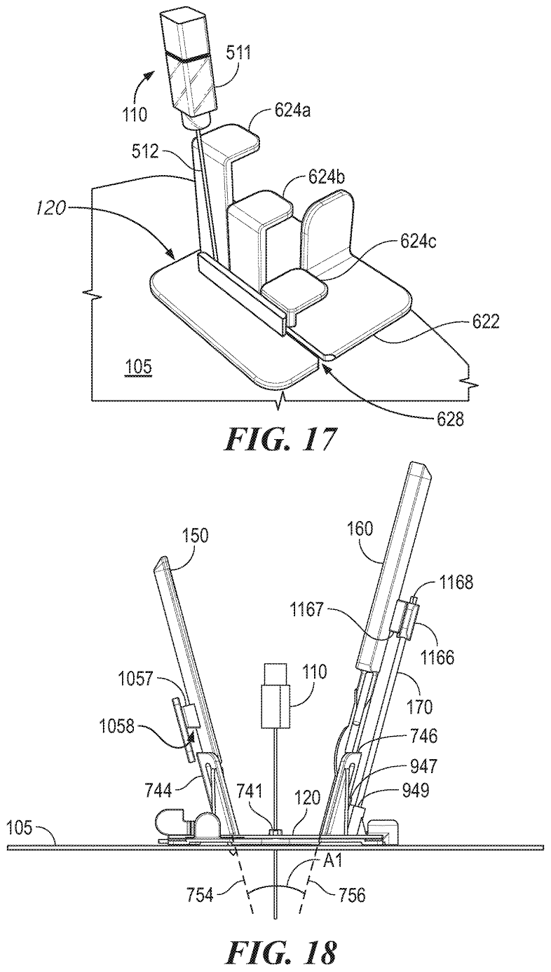

Additionally, the working port scalpels 160a-160d are labeled “1,” “2,” “3,” and “4,” respectively. As discussed in further detail herein, the working port scalpels 160a-160d can be associated with different sized ones of the angle plate guide 130. For example, as discussed below with reference to FIG. 17 and Table 1, different patients can require different tissue penetration depths, and an appropriately sized angle plate guide 130 and working port scalpel 160 can be selected accordingly. In particular, because the sleeves 1166 are positioned at different distances from the distal tips of the corresponding blade holders 1163, the working port scalpels 160a-160d can be associated with different maximum insertion depths.

The working port scalpel 160e labeled “FD” can be a “free depth” scalpel having its sleeve 1166 positioned farthest from the distal tip of the corresponding blade holder 1163 among the working port scalpels 160a-160e. In some cases, the physician may opt to use the working port scalpel 160e instead of one of the working port scalpels 160a-160d in order to perform the procedure in a “free hand” manner without necessarily being limited by a pre-defined insertion depth. It is appreciated that the five working port scalpels 160a-160e illustrated in FIG. 11C are merely illustrative examples, and that working port scalpels 160 of the present technology have different sizes and/or configurations.

FIGS. 12A and 12B are isometric and bottom views, respectively, of the tissue dissector 180. Referring to FIGS. 12A and 12B together, the tissue dissector 180 can include a handle 1282 and a blade 1284 extending from the handle 1282. As shown, the blade 1284 can have a tapered tip 1285 that facilitates insertion of the tissue dissector 180 into patient tissue. In some embodiments, the blade 1284 has a width D4 less than the gap 748 (FIG. 7A) so that the connector 1167 can fit therein.

FIGS. 13A and 13B are isometric and top views, respectively, of the flag guide 190. Referring to FIGS. 13A and 13B together, the flag guide 190 can include a plate 1392, a pair of wings or legs 1394 coupled to lower ends of either side of the plate 1392, and a protrusion 1396 extending from the plate 1392. The plate 1392 can define a needle channel 1395, a first instrument channel 1398a, and a second instrument channel 1398b. As shown, each of the needle channel 1395, the first instrument channel 1398a, and the second instrument channel 1398b can be an open channel. The needle channel 1395 can be shaped and sized to receive the needle 512 of the spinal needle 110 therethrough. In the illustrated embodiment, the plate 1392 includes bumps 1397 positioned along the needle channel 1395 and sized to more securely receive the needle 512. The first instrument channel 1398a and the second instrument channel 1398b can be shaped and sized to receive, for example, the visualization instrument 104 and the working instrument 106 (FIG. 1), respectively. As discussed in further detail herein with reference to FIG. 16, the angle and the distance between the first instrument channel 1398a and the second instrument channel 1398b can correspond to the angle and the distance between the first guiding ramp 744 and the second guiding ramp 746 (FIG. 7A) of the angle plate guide 130 selected for the particular patient. The flag guide 190 can be composed of plastic, metal, and/or other suitable material, and can be manufactured via molding, additive manufacturing (e.g., 5D printing), machining, and/or other suitable techniques.

The pair of legs 1394 can provide substantially flat surfaces so that the flag guide 190 may stand on the patient's skin. In some embodiments, the pair of legs 1394 are positioned lower than the plate 1392 such that when the pair of legs 1394 are placed on the patient's skin, the plate 1392 remains lifted and does not directly contact the patient's skin. The protrusion 1396 can serve as a handle for manipulating the flag guide 190 and/or a support for the physician's finger.

FIG. 14 is a plan view of a surgical kit 1400 configured in accordance with various embodiments of the present technology. The surgical kit 1400 can include zero, one, or more of any of the components described herein. For example, in the illustrated embodiment, the surgical kit 1400 includes the spinal needle 110, the needle depth gauge 120 (illustrated schematically), the scope port scalpel 150, the guide rod 170, the tissue dissector 180, the visualization instrument 104, and the working instrument 106. The surgical kit 1400 can also include a plurality of angle plate guides 130 (individually labeled 130a-130d), a plurality of working port scalpels 160a-160e, and a plurality of flag guides 190 (individually labeled 190a-190d). The blades of the working port scalpels 160a-160e are omitted for illustrative purposes only. Different ones of the plurality of angle plate guides 130 can have different angles and/or distances between the first guiding ramps 744 and the corresponding second guiding ramps 746 (FIG. 7A). Similarly, different ones of the plurality of flag guides 190 can have different angles and/or distances between the first instrument channels 1398a and the corresponding second instrument channels 1398b. As discussed in further detail herein, the physician can select the appropriately sized angle plate guide 130, working port scalpel 160, and flag guide 190 (e.g., specific to the particular patient) depending on the measurement taken from the needle depth gauge 120. While a total of four angle plate guides 130, five working port scalpels 160a, and four flag guides 190 are included in the illustrated embodiment, the surgical kit 1400 can include fewer or more angle plate guides 130, working port scalpels 160, and/or flag guides 190. The guides and components of the kits can be disposable or reusable.

FIGS. 15 and 16 illustrate operation of the needle guide 113 in accordance with various embodiments of the present technology. Referring first to FIG. 15, once a patient is lying face-down on an operating table, a physician can configure the needle guide 113 such that the rod 119 extends past the distal end 114a, and position the needle guide 113 adjacent to the patient such that the rod 119 aligns with, for example, a target spinal disc 1501 to be removed. Notably, the needle guide 113 is positioned to the side of the patient such that the rod 119 does not pierce the patient's skin, and is merely aligned with the target spinal disc 1501 when viewed from the side. Imaging techniques (e.g., X-ray) can be employed to visualize and confirm that the rod 119 is aligned with the target spinal disc 1501. Subsequently, the physician can mark the aligned position of the needle guide 113 on the patient, such as by using a marker to draw a line on the patient's skin.

Referring next to FIG. 16, the physician can position the needle guide 113 above the target spinal disc 1501 (e.g., by referencing the mark drawn earlier). Specifically, the physician can press the distal end 114a against the patient's skin 105 to stabilize the position of the needle guide 113. The physician can then insert the needle 512 of the spinal needle 110 into the gap 115 between the wings 116 of the needle guide 113 at a desired lateral position. Accordingly, the needle guide 113 can help the physician insert the spinal needle 110 at a position that aligns with a target site (e.g., the target spinal disc 1501).

FIG. 17 illustrates operation of the spinal needle 110 and the needle depth gauge 120 in accordance with various embodiments of the present technology. First, the physician can insert the spinal needle 110 into the patient's skin 105 and towards a target site, as discussed above with reference to FIGS. 15 and 16. For example, the physician can insert the spinal needle 110 until the distal tip of the needle 512 reaches a lamina surface of the patient's spine, which the physician can determine based on tactile feedback, imaging, and/or the like.

Second, the physician can slide the needle depth gauge 120 over the patient's skin 105 such that the slot 628 receives and moves relative to the stationary spinal needle 110. As the needle depth gauge 120 moves relative to the spinal needle 110, the handle 511 of the spinal needle 110 may hit or abut against the base 622 or one of the depth indicators 624a-624c of the needle depth gauge 120. For example, if the spinal needle 110 is inserted relatively deep in the patient, the handle 511 may abut against the shortest depth indicator 624c such that the handle 511 is positioned over the marking “4.” If the spinal needle 110 is inserted relatively shallow in the patient, the handle 511 may go past all of the depth indicators 624a-624c such that the handle 511 is positioned over the marking “1” (as shown in FIG. 17). Accordingly, the needle depth gauge 120 can be used to measure the approximate depth of the spinal needle 110 in the patient. Table 1 below provides example measurements corresponding to each of the markings “1,” “2,” “3,” and “4” on the needle depth gauge 120.

TABLE 1

| Marking on | Tissue | Angle plate guide | Cannula |
|---|---|---|---|
| the needle | Depth | insertion | length |
| depth gauge | (mm) | depth (mm) | (mm) |
| 1 | 30-45 | 38 | 30, 40 |
| 2 | 46-60 | 53 | 50, 60 |
| 3 | 61-80 | 70 | 60, 70, 8 |
| 4 | 81-100 or more | 90 | 90, 100 |

The second column of Table 1 indicates the approximate tissue depth between the patient's skin 105 and the patient's spine. The third column of Table 1 indicates the depth at which instruments guided along the first guiding ramp 744 and the second guiding ramp 746 of the angle plate guide 130 would meet within patient tissue. As discussed above with reference to FIG. 14, different angle plate guides 130 of different sizes may be available for the physician to select from. For example, different angle plate guides 130 may include the first guiding ramps 744 and the second guiding ramps 746 at the same angle (e.g., 50 degrees relative to one another), but spaced apart by different distances. The fourth column of Table 1 indicates the appropriate cannula length. In some embodiments, the specific cannula length may be selected further based on, for example, dilator measurements, physician preference, and/or the like. It is appreciated that the values listed in Table 1 are merely illustrative examples, and that the different markings on the needle depth gauge 120 can be paired with other tissue depths/ranges.

Accordingly, depending on which marking on the needle depth gauge 120 that the handle 511 of the spinal needle 110 is positioned above, the physician can select the appropriate angle plate guide 130 (e.g., among the plurality of angle plate guides 130a-130d of FIG. 14), the appropriate working port scalpel 160 (e.g., among the plurality of working port scalpels 160a-160e), and the appropriate flag guide 190 (e.g., among the plurality of plate guides 190a-190d of FIG. 14). The angle plate guides 130, the working port scalpels 160, and the flag guides 190 can be marked differently (e.g., with corresponding markings “1,” “2,” “3,” and “4”) and/or color-coded differently.

FIG. 18 illustrates operation of the angle plate guide 130, the scope port scalpel 150, and the working port scalpel 160 in accordance with various embodiments of the present technology. Once the physician has selected the appropriately sized angle plate guide 130 and working port scalpel 160, as discussed above, the physician can position the selected angle plate guide 130 on the patient's skin 105. As discussed above with reference to FIG. 7B, the physician can position the angle plate guide 130 in the open state around the spinal needle 110, transition the angle plate guide 130 to the closed state (e.g., using the latch 739) such that the spinal needle 510 is received in the aperture 741, and optionally secure the angle plate guide 130 to the patient's skin 105 via the one or more apertures 731 (FIG. 7A).

Next, the physician can form a first port and a second port (e.g., in sequence) using the scope port scalpel 150 and the selected working port scalpel 160, respectively. When using the scope port scalpel 150, the physician can position and slide the scope port scalpel 150 along the first guiding ramp 744 until the gap 1058 receives and the connector 1057 abuts against the first guiding ramp 744. Therefore, (i) the position of the connector 1057 along the length of the scope port scalpel 150 and (ii) the height of the first guiding ramp 744 can define a maximum insertion depth of the scope port scalpel 150, preventing accidentally inserting the scope port scalpel 150 too deep. When using the working port scalpel 160, the physician can position and slide the working port scalpel 160 along the second guiding ramp 746. Also, the physician can align the sleeve 1166 with the guide rod 170 such that the guide rod 170 is received in the channel 1168 of the sleeve 1166. The guide rod 170, received in the recess 949, can ensure that the working port scalpel 160 pierces the patient's skin 105 along a straight axis. The physician may continue to insert the working port scalpel 160 until the connector 1167 abuts against the stop 947 of the second guiding ramp 746. Therefore, (i) the position of the connector 1167 along the length of the working port scalpel 160 and (ii) the position of the stop 947 can define a maximum insertion depth of the working port scalpel 160, preventing accidentally inserting the working port scalpel 160 too deep. The size of the scope port (e.g., the incision formed by the scope port scalpel 150) and the size of the working port (e.g., the incision formed by the working port scalpel 160) can be between about 4-5 mm and between about 9-10 mm, respectively.

As shown, the scope port scalpel 150 and the working port scalpel 160 can be oriented relative to one another by the angle A1 defined by the axes 754, 756. Therefore, (i) the access path extending from the first port (also referred to herein as “the scope port”) formed by the scope port scalpel 150 and (ii) the access path extending from the second port (also referred to herein as “the working port”) formed by the working port scalpel 160 can also be oriented at the angle A1 relative to one another and can extend toward the working space in the patient.

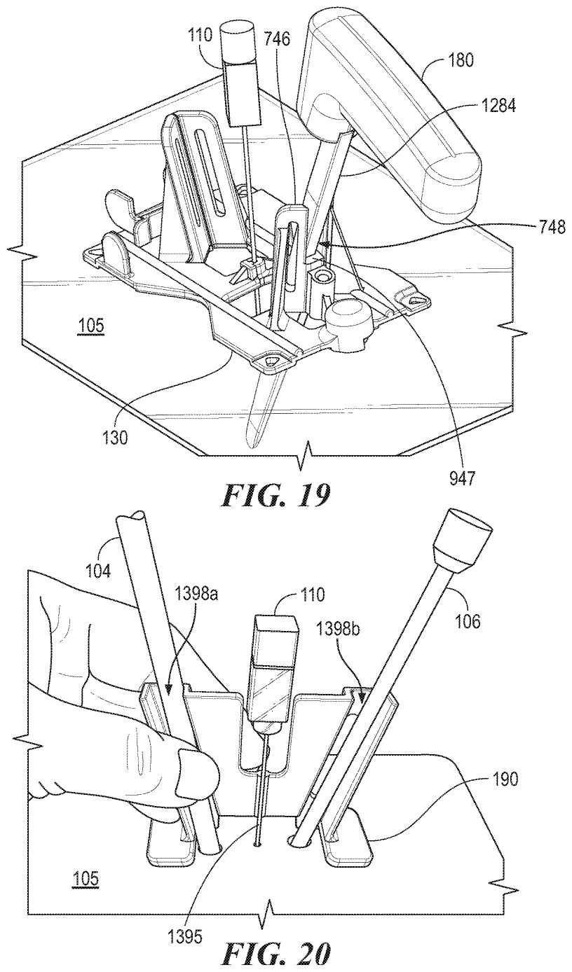

FIG. 19 illustrates operation of the angle plate guide 130 and the tissue dissector 180 in accordance with various embodiments of the present technology. After forming the scope port, the working port, and their corresponding access paths as discussed above, the physician can remove each of the scope port scalpel 150 and the working port scalpel 160, position the tissue dissector 180 along the second guiding ramp 746, and insert the blade 1284 of the tissue dissector 180 into the working port. As shown, the blade 1284 can fit within the gap 748 of the second guiding ramp 746. Accordingly, the physician may pivot the tissue dissector 180 against, for example, an edge of the stop 947 to enlarge the access path extending from the working port.

FIG. 20 illustrates operation of the flag guide 190 in accordance with various embodiments of the present technology. Once the access paths have been formed and enlarged, as discussed above, the physician can remove the angle plate guide 130 from the patient's skin 105, then insert the visualization instrument 104 and the working instrument 106 into the scope port and the working port, respectively. In other embodiments, the angle plate guide 130 can remain on the patient and the visualization instrument 104 and the working instrument 106 (e.g., guided by associated cannulas) can be inserted through the first opening 736 and the second opening 738 (FIG. 7A), respectively. The spinal needle 110 may remain in or be removed from the patient's skin 105 during operation. Subsequently, while operating on the patient, the physician may choose to confirm whether the visualization instrument 104 and the working instrument 106 are angled properly. For example, while the visualization instrument 104 and the working instrument 106 are inserted through the scope port and the working port, respectively, the physician can select and slide the appropriately sized flag guide 190 across the patient's skin 105 until (i) the needle channel 1395 receives the spinal needle 110, (ii) the first instrument channel 1398a receives the visualization instrument 104, and (iii) the second instrument channel 1398b receives the working instrument 106. The flag guide 190 can remain in the illustrated position during operation, or be removed once the physician has confirmed the proper angling of the visualization instrument 104 and the working instrument 106.

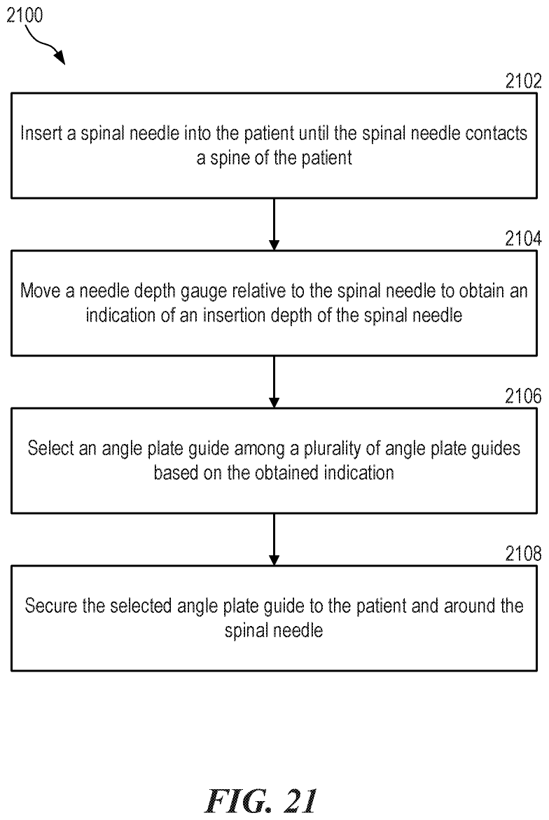

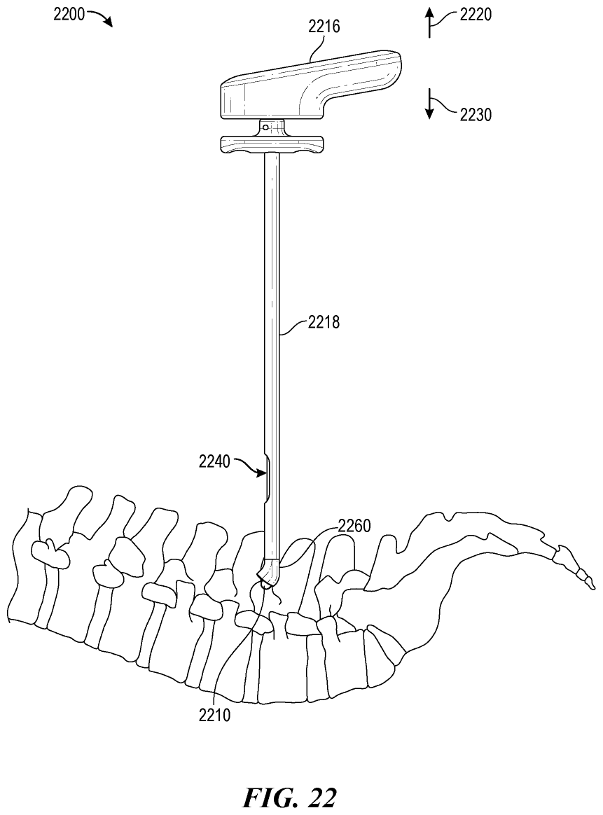

FIG. 21 is a flowchart illustrating a method 2100 for performing a multi-portal spine procedure in accordance with various embodiments of the present technology. While the steps of the method 2100 are described below in a particular order, one or more of the steps can be performed in a different order or omitted, and the method 2100 can include additional and/or alternative steps. Additionally, although the method 2100 may be described below with reference to the embodiments of the present technology described herein, the method 2100 can be performed with other embodiments of the present technology.

The method 2100 begins at block 2102 by inserting a spinal needle into the patient until the spinal needle contacts a spine of the patient. In some embodiments, the spinal needle is inserted until a distal tip of the spinal needle contacts a lamina surface of the spine, is positioned at a targeted depth, position, etc. The distal tip can be viewed via, for example, fluoroscopy or other imaging techniques.

At block 2104, the method 2100 continues by moving a needle depth gauge relative to the spinal needle to obtain an indication of an insertion depth of the spinal needle. In some embodiments, moving the needle depth gauge comprises receiving the spinal needle in a slot of the needle depth gauge and sliding the needle depth gauge until the spinal needle (i) abuts against one of a plurality of depth indicators of the needle depth gauge or (ii) is positioned above a tallest one of the plurality of depth indicators.

At block 2106, the method 2100 continues by selecting an angle plate guide among a plurality of angle plate guides based on the obtained indication. In some embodiments, each of the plurality of angle plate guides includes first and second guiding ramps, and different ones of the plurality of angle plate guides have their respective first and second guiding ramps spaced apart by different distances.

At block 2108, the method 2100 continues by securing the selected angle plate guide to the patient and around the spinal needle. In some embodiments, securing the selected angle plate guide comprises transitioning the selected angle plate guide from an open state to a closed state.

In some embodiments, the method 2100 continues further by (i) sliding a scope port scalpel along a first guiding ramp of the selected angle plate guide and (ii) sliding a working port scalpel along a second guiding ramp of the selected angle plate guide. Sliding the working port scalpel can comprise sliding a sleeve of the working port scalpel along a guide rod coupled to the selected angle plate guide.

In some embodiments, the method 2100 continues further by maneuvering a tissue dissector along a guiding ramp of the selected angle plate guide. The guiding ramp can include a gap sized to fit a blade of the tissue dissector therein.

In some embodiments, the method 2100 continues further by (i) forming a first access path and a second access path in the patient via a first scalpel and a second scalpel, respectively, each guided by the selected angle plate guide, (ii) inserting a visualization instrument in the first access path, and (iii) inserting a working instrument in the second access path. The method 2100 may continue by moving a flag guide across the patient until (i) a needle channel of the flag guide receives the spinal needle, (ii) a first instrument channel of the flag guide receives the visualization instrument, and (iii) a second instrument channel of the flag guide receives the working instrument.

One or more steps of the method 2100 can be repeated during the surgical procedure to use different guides to perform different surgical actions at different locations within the patient.

### III. Select Embodiments of Balloon Dissectors

FIG. 22 is a side view of a bone wax applicator 2200 positioned along a human patient's spine. The bone wax applicator 2200 can be used to controllably dispense bone wax 2210 to sites of bleeding (e.g., surfaces of bony anatomy, tissue, etc.). The bone wax applicator 2200 can include a driver assembly 2216 and a dispensing assembly 2218. The driver assembly 2216 can be moved proximately, as indicated by arrow 2220, to load bone wax through a loading window 2240 and can then be moved distally, as indicated by arrow 2230, to push the bone wax 2210 out of a distal tip 2260. The bone wax applicator 2200 can be used to apply one or more of bone wax, hemostatic agents (e.g., liquids, gels, etc.), adhesives, medicants, or other materials.

The bone wax applicator 2200 can be delivered through cannulas, endoscope ports, or access instruments or openings. In endoscopic procedures, the bone wax can be used to staunch bleeding from bone and can be applied during continuous irrigation or when irrigation is temporarily stopped. The bone wax applicator 2200 can be used in single-port procedures or multi-portal procedures (e.g., dual-portal endoscopic procedures). In open procedures, the bone wax applicator 2200 can be used to staunch bleeding from bone structures under direct viewing. The configuration of the bone wax applicator 2200 and substance to be applied can be selected based on the surgery and surgical steps to be performed. The bone wax can be applied to the injured tissue by moving the atraumatic distal tip along the site. A user can concurrently dispense wax while moving the bone wax applicator 2200. After dispensing, the process can be repeated to reload the bone wax applicator and to reapply the bone wax. This process can be performed any number of times during the procedure to inhibit, limit, or substantially prevent bleeding.

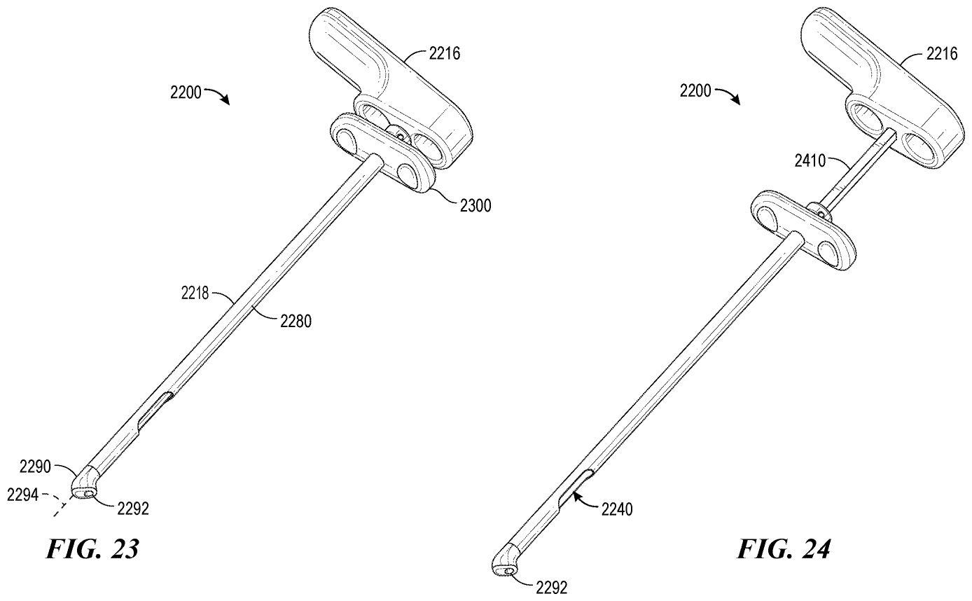

FIG. 23 is an isometric view of the bone wax applicator 2200 in accordance with at least some embodiments of the present technology. The dispensing assembly 2218 includes a cylinder, shaft, or tubular main body 2280 and a curved distal tip 2290 with a dispensing opening 2292 angled to dispense bone wax away from a longitudinal axis 2294 of the tubular main body 2280. The dispensing assembly 2218 can have a gripping member 2300 that can be used by a user to help pull the driver assembly 2216 distally. In some procedures, the tip 2290 can be replaced with another tip (e.g., tip 2260 of FIG. 22).

FIG. 24 is an isometric view of the bone wax applicator 2200 in a loading configuration. A user can load bone wax via the open loading window 2240. The driver assembly 2216 can include a plunger piston or shaft 2410 (“plunger shaft 2410”) that slides along the tubular main body 2280 to drive the bone wax material distally away from the window 2240 toward the opening 2292.

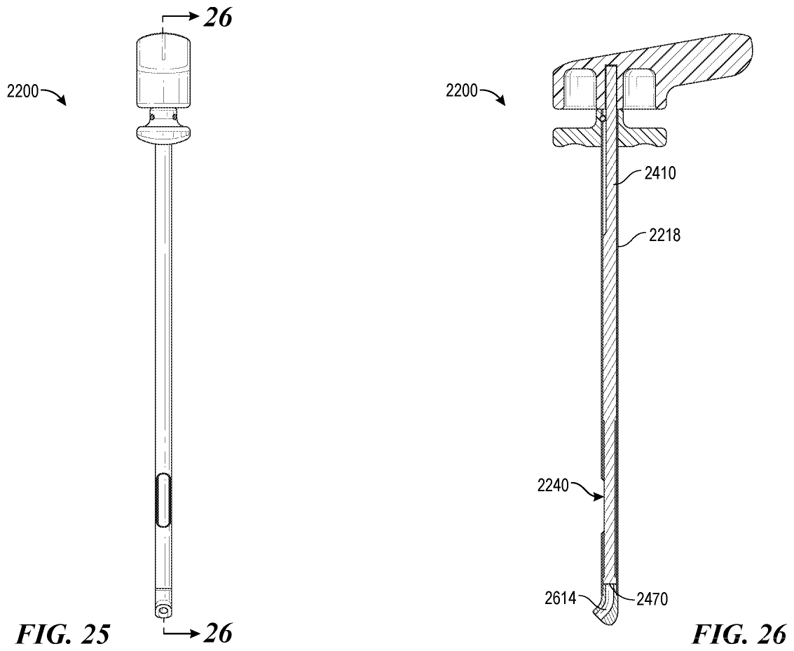

FIG. 25 is a top-plan view of the bone wax applicator 2200. FIG. 26 is a cross-sectional view of the bone wax applicator 2200 taken along line 26-26 of FIG. 25. Referring to FIG. 26, the shaft 2410 that extends along a passageway of the dispensing assembly 2218. The distal end 2470 of the shaft 2410 and the inner surface of the dispensing assembly 2218 can form, for example, a seal (e.g., a liquid tight seal, a gel-tight seal, fluid-tight seal, etc.) such that the distally advancing distal end 2610 pushes wax (e.g., liquified wax, solid wax, etc.) distally along an interior passageway 2614.

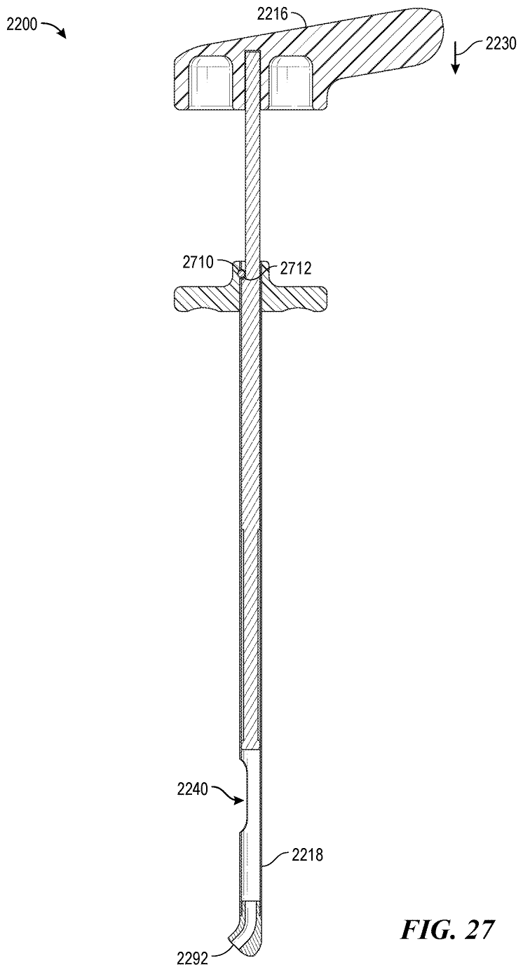

FIG. 27 is a cross-sectional view of the bone wax applicator 2200 taken along line 26-26 of FIG. 25 in the loading configuration. As shown, the gripping member 2300 can include a stopper 2710 and the dispensing assembly 2218 can include a notch 2712 that can engage a bottom side of the stopper 2710. In operation, physical contact between the stopper 2710 and the notch 2712 can prevent the driver assembly 2216 from being pulled too far away from the gripping member 2300.

### IV. Select Embodiments of Endoscope Holders

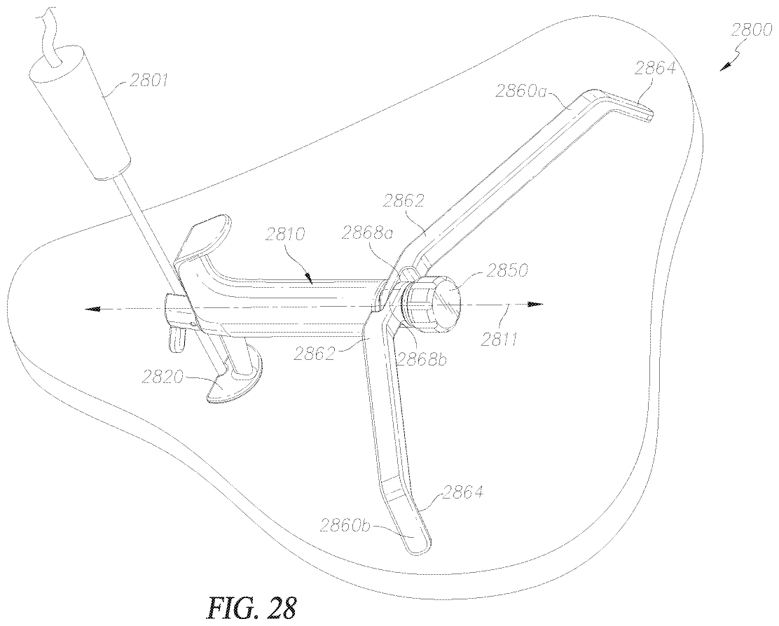

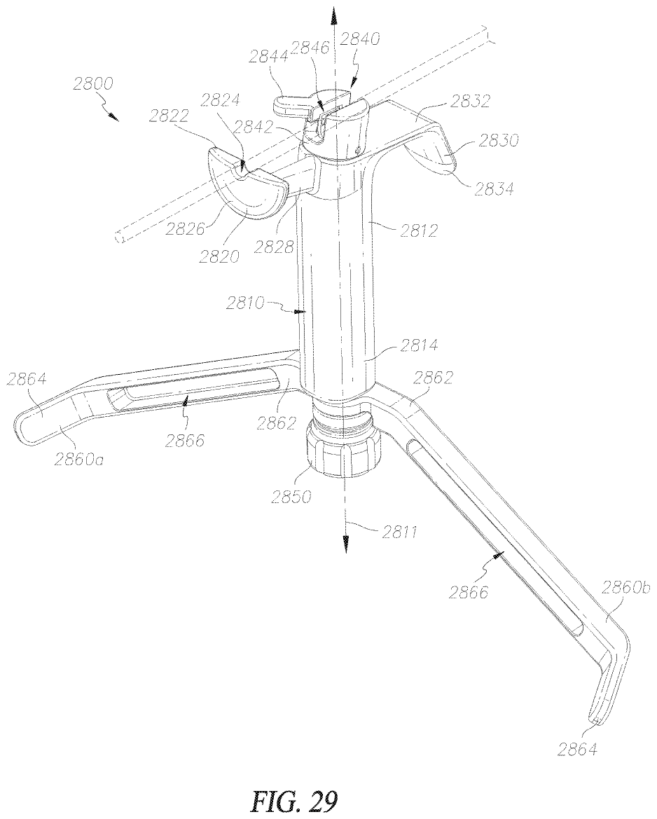

FIG. 28 shows the endoscope holder 2800 holding an endoscope 2801 in a patient in accordance with embodiments of the present technology. FIG. 29 is a bottom-front isometric view of an endoscope holder 2800 in a first position in accordance with embodiments of the present technology. Referring now to FIG. 28, the endoscope holder 2800 can be used to, for example, reduce hand fatigue, reduce inadvertent movement in an endoscope 2801 due to surgeon fatigue, assist with endoscope positioning, or the like. The endoscope holder 2800 can selectively grip and hold the endoscope stationary for viewing instruments inside the patient. In spinal procedures, the patient's spine can be visualized using the endoscope 2801 to view, for example, vertebral spacing, vertebral alignment, tissue (e.g., damaged or displaced sections of intervertebral cartilage disc, tissue contributing to nerve compression, etc.), instruments and implants before, during, and after implantation, or the like. The visualization can help a surgeon throughout the surgical procedure to improve patient outcomes.

Referring now to FIG. 29, the endoscope holder 2800 can include a grip or handle 2810. The handle 2810 can include a proximal portion 2812 and a distal portion 2814. At the proximal portion 2812, the handle 2810 can include a stabilizer foot 2820, a top guard 2830, and a cam assembly 2840. In the illustrated embodiment, the stabilizer foot 2820 and the top guard 2830 are integrally formed with the handle 2810. In other embodiments, the stabilizer foot 2820 and the top guard 2830 can be removably coupled to the handle 2810, for example, by snap fit. In the illustrated embodiment, the cam assembly 2840 is affixed to the handle 2810 such that the cam assembly 2840 can be rotated around an axis 2811 (e.g., a longitudinal axis of the of the handle 2810).

The stabilizer foot 2820 is configured to rest on a patient. In some embodiments, stabilizer foot 2820 is semicircular and includes an upper surface 2822. The upper surface 2822 of the stabilizer foot 2820 defines a window or semicircular recess 2824 that is concentric with the outer diameter of the stabilizer foot 2820. The semicircular recess 2824 can be sized to slideably receive a shaft (illustrated in phantom) of the endoscope. Further, the outer surface 2826 of the stabilizer foot 2820 can be rounded to help cushion tissue, thereby limiting or preventing tissue injury. The semicircular shape allows for viewing a portion of the port and helps the stabilizer foot 2820 to distribute weight from the endoscope holder 2800 across a larger area and it also allows a user to easily remove an endoscope from the top of the endoscope holder 2800. Between the outer surface 2826 and the cam assembly 2840 is a narrow portion or support 2828 configured to reduce the bulk of the endoscope holder 2800 and to enhance the ergonomics of the endoscope holder 2800. In some embodiments, the support 2828 has a fixed length. In some embodiments, the support 2828 is extendable (e.g. via telescoping mechanisms) to allow adjustment of the height to the handle 2810.

The top guard 2830 includes a flat portion 2832 adjacent to the cam assembly 2840 and a bent portion 2834 adjacent to the flat portion 2832. Together, the flat portion 2832 and the bent portion 2834 are configured to wrap over the user's hand between the thumb and forefinger. For example, the bent portion 2834 can rest against the back of the user's hand to provide stability and prevent inadvertent contact with the endoscope when an endoscope is placed in the endoscope holder 2800. In the illustrated embodiment, the distance between the bent portion 2834 and the outer surface 2826 of the stabilizer foot 2820 is sized to allow the user to easily grasp the handle 2810 with their hand with their forefinger extending from the top guard 2830 to the semicircular portion of the stabilizer foot 2820. Further, the user's thumb can rest along the opposite side of the handle 2810 and the support 2828. Thus, the handle 2810 acts as a grip, with the user's palm surrounding the handle 2810 and their pinky placed at the distal portion 2814 of the handle 2810. In some embodiments, the handle 2810 can be shaped to include ergonomic features such as indents (e.g., recesses, curves) configured to align with the user's individual fingers.

In the illustrated embodiments, the cam assembly 2840 includes a pivoting holder or cam body 2842 and a trigger 2844. The cam body 2842 defines a slot 2846. The slot 2846 is configured to house, hold, and/or grip the shaft of an endoscope. In the first position, the slot 2846 aligns with the semicircular recess 2824. For example, an endoscope placed into the endoscope holder 2800 could extend from the slot 2846 to the semicircular recess 2824 and beyond. The trigger 2844 is an elongated member extending from the top of the cam body 2842 of the cam assembly 2840. As such, the trigger 2844 can be easily actuated by the forefinger of the user. When the trigger 2844 is actuated (e.g., pressed forward by the user's forefinger), the cam assembly 2840 rotates around the axis 2811 of the handle 2810. The cam body 2842 can further include one or more biasing members configured to keep a semicircular bottom of the slot 2846 of the cam assembly 2840 aligned (e.g., concentrically aligned when viewed from above) with the semicircular recess 2824. As such, when the user releases the trigger 2844 (e.g., stops pressing the trigger 2844), the cam assembly 2840 will return to the position where the semicircular bottom of the slot 2846 is aligned with the semicircular recess 2824. In other embodiments, the cam body 2842 does not include a biasing member and the user must use the trigger 2844 to rotate the cam assembly 2840.

In the illustrated embodiment, the endoscope holder 2800 further comprises a knob 2850 and a pair of stabilizer wings or legs 2860 (individually labeled a first stabilizer leg 2860a and a second stabilizer leg 2860b). The knob 2850 includes an attachment feature (e.g., a threaded post, a threaded hole) that can be attached to an attachment feature of the handle 2810 (e.g., a threaded hole, a threaded post). As such, the knob 2850 can be moved along the axis 2811 of the handle 2810, thereby extending or reducing the overall length of the endoscope holder 2800, locking the stabilizer legs 2860, etc. The knob 2850 can include a rounded bottom surface to help slide along tissue, thereby limiting or preventing tissue injury. The knob 2850 can be used by the user to adjust the endoscope holder 2800 to varying workspace needs and to maintain the stability of the endoscope holder 2800 throughout the procedure when used as an additional stabilizer. In other embodiments, the knob 2850 can be permanently attached to the handle 2810 or can be excluded entirely.

In the illustrated embodiment, the stabilizer legs 2860 each have a proximal portion 2862 and a distal portion 2864. At the proximal portions 2862, the stabilizer legs 2860 each define an opening (not shown) through which the attachment feature of the knob 2850 can be extended (e.g., threaded). As such, the stabilizer legs 2860 can be stacked on the attachment feature of the knob 2850 and removably coupled to the handle 2810. Thus, in some embodiments, the stabilizer legs 2860 can be removed and the knob 2850 can be reattached and used alone as a stabilizing mechanism for the endoscope holder 2800. The knob 2850 can further include grooves, texturing, or features (e.g., multiple recesses) that enable the user to easily grasp the knob 2850 and engage/disengage the attachment features, for example, by turning the knob 2850.

The stabilizer legs 2860 can further define holes 2866 located along a length of the stabilizer legs 2860. In the illustrated embodiment, the holes 2866 are located between the proximal portions 2862 and the distal portions 2864, however, in other embodiments, the holes 2866 can be located at the proximal portions 2862 and/or the distal portions 2864 or can be excluded altogether. The holes 2866 can be configured to reduce the overall weight of the endoscope holder 2800. The stabilizer legs are also bent at the distal portions 2864, which can help the stabilizer legs contact the skin of the patient and provide support to the endoscope holder 2800, as described in further detail below. The endoscope holder 2800 and its individual components (e.g., the handle 2810, the stabilizer legs 2860) can be made of plastic, surgical steel, and/or other substances suited for sterile surgical environments.

Referring again to FIG. 28, the stabilizer legs 2860 include complimentary recessed portions 2868 (individually labeled a first recessed portion 2868a and a second recessed portion 2868b) located at the proximal portions 2862 of each stabilizer leg 2860. The first recessed portion 2868a defines a recess on the bottom of the first stabilizer leg 2860a and the second recessed portion 2868b defines a recess on the top of the second stabilizer leg 2860b. As such, the recessed portions 2868 can nest to reduce the height of the pair of stabilizer legs 2860 when they are placed on the attachment feature of the handle 2810 and/or the knob 2850. For example, if the first and second recessed portions 2868a and 2868b are half the width of the first and second stabilizer legs 2860a and 2860b at the distal portions 2864, then the height of the stabilizer legs 2860 adjacent to the handle 2810 will be reduced by half. This can reduce the overall length of the endoscope holder 2800 and allow the user more flexibility (e.g., more possible lengths of the endoscope holder 2800) when adjusting the location of the knob 2850 along the axis 2811 of the handle 2810 via the attachment features. In other embodiments, washers and/or the like can be used to vary the locations of the stabilizer legs 2860 along the length of the attachment feature. For example, the first stabilizer leg 2860a could be coupled directly to the handle 2810, and then multiple washers can be placed on the attachment feature to place the second stabilizer leg 2860b away from the first stabilizer leg 2860a and the handle 2810.

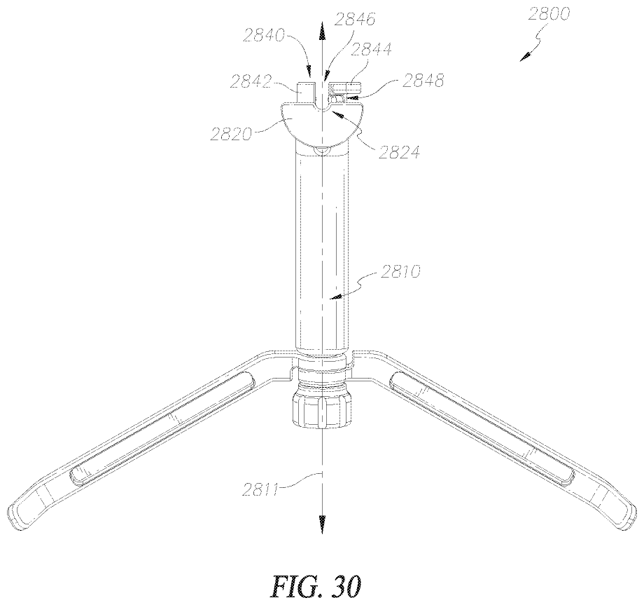

FIG. 30 is a bottom view of the endoscope holder 2800 of FIG. 28 in accordance with embodiments of the present technology. As described above, the handle 2810 includes the stabilizer foot 2820 and the cam assembly 2840. As illustrated, the semicircular recess 2824 aligns with a semicircular bottom of the slot 2846 to create a continuous semicircular cradle for the endoscope. Further, the central axis of the semicircular recess 2824 is aligned (e.g., intersecting) with the axis 2811 of the handle 2810 to align the endoscope with the center of the user's grip, making the endoscope holder 2800 easier to use.

The cam body 2842 of the cam assembly 2840 further defines a slot (e.g., U-shaped slot) or first recess 2848. The first recess 2848 is shaped such that the profile of the bottom of cam body 2842 is maintained when the cam assembly 2840 is rotated by a slight degree (e.g., 5 degrees; the endoscope holder 2800 is in a second position) by the trigger 2844. In other words, when the cam assembly 2840 is rotated by 5 degrees, the view from the front of the semicircular recess 2824 would remain unobstructed through the bottom of the cam assembly 2840. As such, an endoscope held within the slot 2846 would not turn with a slight rotation of the cam assembly 2840. In other embodiments, the first recess 2848 can be shaped such that the profile is maintained at other angles, such as 10 degrees, 15, degrees, 20 degrees, 25 degrees, 30 degrees, 35 degrees, 40 degrees, 45 degrees, or any degree therebetween. In the illustrated embodiment, the profile of the first recess 2848 (e.g., the semicircular diameter) is not concentrically aligned with the semicircular recess 2824.

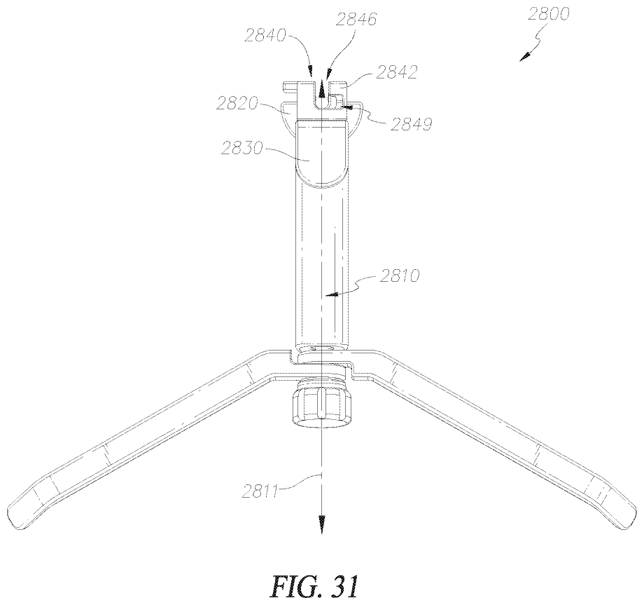

FIG. 31 is a top view of the endoscope holder 2800 of FIG. 28 in accordance with embodiments of the present technology. As described above, the handle 2810 includes the stabilizer foot 2820, the top guard 2830, and the cam assembly 2840. In the illustrated embodiment, the cam body 2842 of the cam assembly 2840 defines a second recess 2849. The second recess 2849 mirrors the first recess 2848 such that the combination of the first and second recesses 2848 and 2849 creates a continuous cylindrical hole through the cam body 2842 of the cam assembly 2840. A center of the cylindrical hole (e.g., a center of a circular cross-section of the cylindrical hole) created by the first and second recesses 2848 and 2849 is at the same location along the axis 2811 as a center of the semicircular bottom of the slot 2846 (e.g., a center of a semicircular cross-section of the slot 2846).

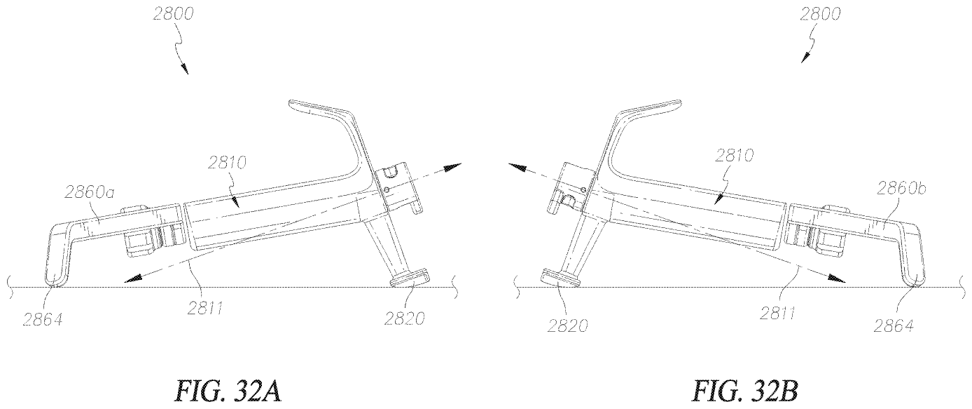

FIGS. 32A and 32B are side views of the endoscope holder 2800 of FIG. 28 positioned along a patient in accordance with embodiments of the present technology. As described above, the endoscope holder 2800 includes the pair of stabilizer legs 2860. More specifically, FIG. 32A shows the first stabilizer leg 2860a and FIG. 32B shows the second stabilizer leg 2860b. With reference to FIG. 32A, the distal portion 2864 of the first stabilizer leg 2860a extends in the same direction as the stabilizer foot 2820. With reference to FIG. 32B, the distal portion 2864 of the second stabilizer leg 2860b extends in the same direction as the stabilizer foot 2820.

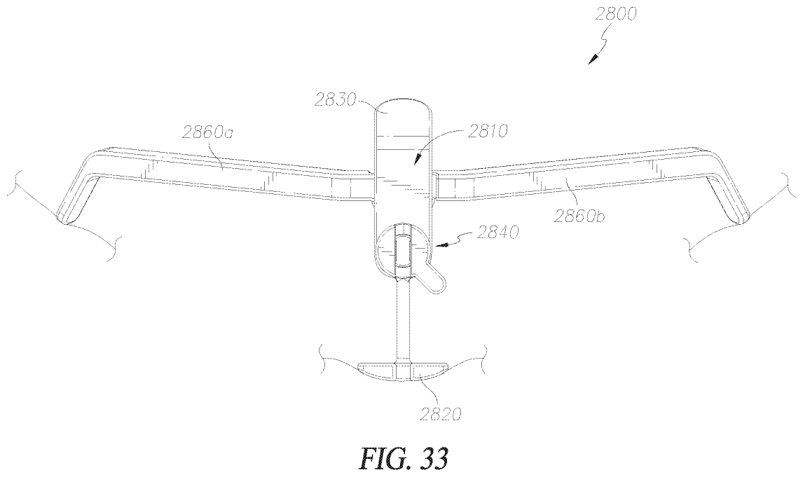

FIG. 33 is a front view of the endoscope holder 2800 of FIG. 28 resting on a patient in accordance with embodiments of the present technology. As described above, the endoscope holder 2800 includes the handle 2810 and the stabilizer legs 2860. The handle 2810 also includes the stabilizer foot 2820, the top guard 2830, and the cam assembly 2840.

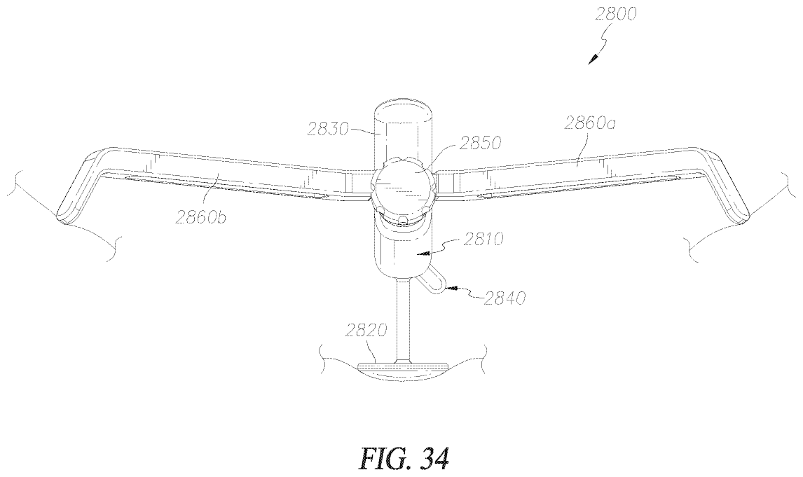

FIG. 34 is a back view of the endoscope holder 2800 of FIG. 28 resting on a patient in accordance with embodiments of the present technology. As described above, the endoscope holder 2800 includes the handle 2810, the knob 2850, and the stabilizer legs 2860. The handle 2810 also includes the stabilizer foot 2820, the top guard 2830, and the cam assembly 2840.

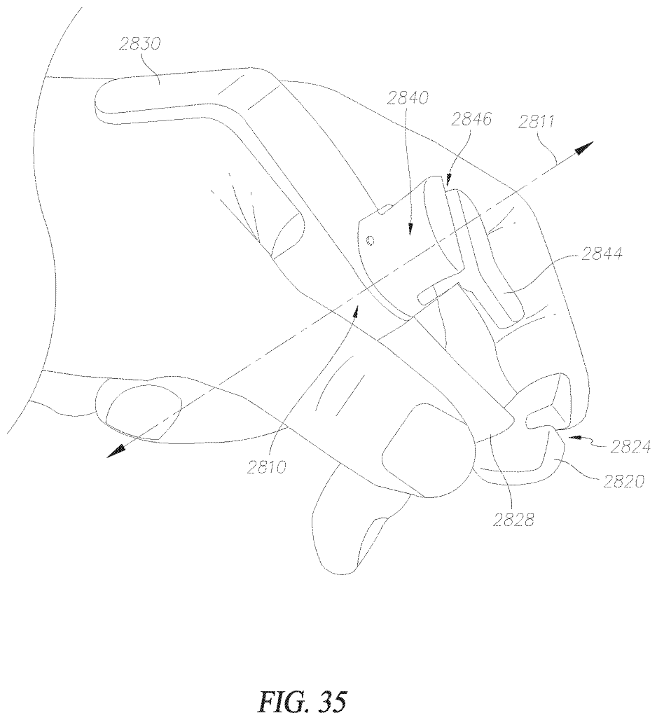

FIG. 35 is an isometric view of a portion of the endoscope holder 2800 of FIG. 28 in a second position in accordance with embodiments of the present technology. As described above, the handle 2810 includes the stabilizer foot 2820, the top guard 2830, and the cam assembly 2840. In the illustrated embodiment, the user grasps the handle 2810 such that their forefinger rests against the stabilizer foot 2820 and their thumb rests along the side of the handle 2810 and the support 2828. Further, the top guard 2830 rests on the back of the user's hand, distributing the weight of the endoscope holder 2800 across the back of the user's hand. Other users may rest their fingers in other locations, for example, a user may rest their forefinger along the support 2828. The shape of the endoscope holder 2800 allows for a variety of grips depending on user preferences.

In the illustrated embodiment, the endoscope holder 2800 is in the second position. As such, the trigger 2844 is actuated (e.g., pressed forward) so that the cam assembly 2840 rotates around the axis 2811 of the handle 2810. The rotation of the cam assembly 2840 changes the orientation of the slot 2846 such that the semicircular bottom is not concentrically aligned with the semicircular recess 2824. Instead, the semicircular profiles of the first and second recesses 2848 and 2849 are concentrically aligned with the semicircular recess 2824. In this configuration, an endoscope housed with the slot 2846 would remain concentrically aligned with the semicircular recess 2824 and would be squeezed (e.g., held in place by friction) by opposing walls of the first and second recesses 2848 and 2849. As such, the second position acts as a locking position to hold the endoscope in place. In other embodiments, the cam assembly 2840 can include additional features that activate when the cam assembly 2840 is rotated to lock the endoscope into place.

During the operation of the endoscope holder 2800, the user could repeatedly alternate between the first and second positions, allowing the user to move the endoscope holder 2800 along the length of the endoscope as needed. For example, the user could be holding an endoscope at a first length in the second position. When the user wishes to move to a second length, they could release the trigger 2844, returning the cam assembly 2840 to the first position and releasing the endoscope. The user could then move to the second length and actuate the trigger 2844 to lock the endoscope into place at the second length.

Referring again to FIG. 28, the stabilizer legs 2860 are used to stabilize the endoscope holder 2800. To do so, they rest on the patient's abdomen. Further, the stabilizer foot 2820 (not shown) rests against the patient's skin, stabilizing the endoscope holder 2800. In effect, the stabilizer legs 2860 and the stabilizer foot 2820 create a tripod that rests against the patient's skin to stabilize the endoscope. This configuration allows the user of the endoscope holder 2800 to place the weight of the endoscope on the patient through the stabilizer legs 2860 and the stabilizer foot 2820. While endoscopes are light enough not to cause harm to a patient when placed on the skin, holding them for extended periods can strain a surgeon's hands and arms. More specifically, since endoscopes are long and narrow, surgeons typically pinch the endoscope between their fingers to hold it, which can cause pain when held for extended periods. As such, distributing the weight to the patient's skin reduces the strain on the surgeon's arms and hands without creating risk for the patient.

Embodiments of the present technology can reduce the strain on surgeons who use endoscopes, which can increase the possible length of procedures and prevent injuries in surgeons from recurrent strain. Further, placing the endoscope in a holder can reduce accidental movement in the endoscope due to surgeon fatigue, and in turn, this can reduce the amount of accidental trauma to non-target tissue, such as the tissue surrounding the port through which the endoscope extends. The endoscope holder includes an intuitive mechanism for locking the endoscope into place to ensure that the endoscope does not slide back and forth in the endoscope holder. The stabilizers also include blunt, smooth, and/or curved surfaces and edges, minimizing the risk of injury to the patient and ensuring safety during the use and manipulation of the endoscope holder. Overall, endoscope holders configured in accordance with embodiments of the present technology reduce fatigue in surgeons' arms and hands, allowing procedures to last longer and reducing errors in maneuvering the endoscope due to fatigue.

### V. Select Embodiments of Balloon Dissectors

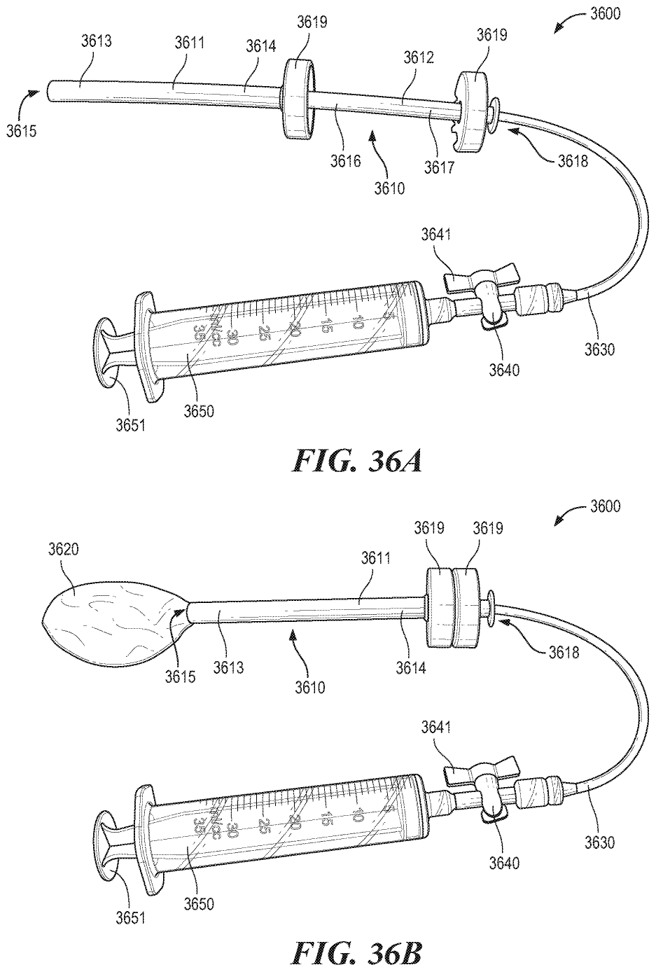

FIGS. 36A and 36B are side views of a spinal surgical system 3600 in an undeployed position and a deployed position, respectively, in accordance with at least some embodiments of the present technology. With reference to FIGS. 36A and 36B, the system can include a dissector device 3610 configured to deploy an expandable dissector 3620 (e.g., a balloon, an inflatable dissector). The dissector device 3610 can include a housing 3611 and a plunger 3612, each having an elongate tubular shape. The plunger 3612 can have an outer diameter smaller than an inner diameter of the housing 3611. The housing 3611 can have a first proximal portion 3613, a first distal portion 3614, and a first lumen 3615 extending therethrough. The plunger 3612 can include a second proximal portion 3616, a second distal portion 3617, and a second lumen 3618 extending therethrough. The second proximal portion 3616 of the plunger 3612 can be inserted into the first lumen 3615 of the housing 3611 at the first distal portion 3614 and slidably positioned within the first lumen 3615. Both the housing 3611 and the plunger 3612 can have a stopper 3619 affixed at the first distal portion 3614 and the second distal portion 3617, respectively. The stoppers 3619 can be configured to control, for example, the maximum depth of the plunger 3612 within the first lumen 3615 (e.g., the maximum range of motion of the plunger within the first lumen 3615). In some embodiments, the housing 3611 and/or the plunger 3612 do not include a stopper 3619.

In the illustrated embodiment of FIG. 36A, the housing 3611 houses the collapsed expandable dissector 3620. The expandable dissector 3620 can be made of polymers, latex, silicone, nylon, and/or the like. The expandable dissector 3620 can be formed to be a specific (e.g., predetermined) size, volume, and/or shape when inflated. For example, the expandable dissector can be spherical or ovular in shape. Specific shapes can be created by sealing multiple pieces of latex, silicone, and/or nylon of varying thickness and/or elasticities together. In the illustrated embodiment, an opening of the expandable dissector 3620 can be affixed to the first proximal portion 3613 of the housing. More specifically, the opening of the expandable dissector 3620 can be circumferentially affixed to the opening of the housing 3611 to fluidly couple the expandable dissector 3620 to the first lumen 3615. In other embodiments, the opening of the expandable dissector 3620 can be sealingly coupled to an interior surface of the first lumen 3615 or the second lumen 3618. In the illustrated embodiment, the expandable dissector 3620 is housed in the first lumen 3615. In other embodiments, the expandable dissector 3620 can be housed in other chambers (e.g., pockets, lumens) adjacent to the first lumen 3615.

The spinal surgical system 3600 can further include a tube 3630 fluidly coupling the dissector device 3610 to a stopcock 3640 and an inflator assembly (e.g., pump) in the form of a syringe 3650. More specifically, the tube 3630 is fluidly coupled to the second lumen 3618 of the plunger 3612 and the first lumen 3615 of the housing 3611 sequentially. In some embodiments, the syringe 3650 can be replaced by different pressure sources, such as a pressurized container/canister, an automated pump, or the like. In the illustrated embodiment, the stopcock 3640 can be configured to be actuated by a user to inhibit fluid flow from the syringe 3650 to the tube 3630 and to allow fluid to flow from the syringe 3650 to the tube 3630. For example, a handle 3641 of the stopcock 3640 can be turned to inhibit fluid flow. The syringe 3650 can be configured to be depressed (e.g., compressed, actuated; the illustrated position) and withdrawn via a handle 3651. Further, the syringe 3650 can be removably coupled to the stopcock 3640 and the stopcock can be removably coupled to the tube 3630. The syringe 3650 can hold either fluid (e.g., water, saline, radiopaque flowable liquid, etc.) or gas (e.g., ambient air).

With reference to FIG. 36A, the dissector device 3610 is shown in an insertion configuration for insertion into a patient. In the insertion configuration, only the second proximal portion 3616 of the plunger 3612 is located within the first lumen 3615 and the expandable dissector 3620 is located within the first lumen 3615 at the first proximal portion 3613 of the housing 3611. With reference to FIG. 36B, the dissector device 3610 is shown in a deployed configuration. In the deployed configuration, the plunger 3612 has been depressed into the first lumen 3615 of the housing by the user via the stopper 3619. The user can either press the stopper 3619 of the plunger 3612 towards the stopper 3619 of the housing 3611 or retract the stopper 3619 of the housing 3611 towards the stopper 3619 of the plunger 3612. As the plunger 3612 is depressed, the plunger 3612 can push out the expandable dissector. In some embodiments, the fluid housed within the second lumen 3618 of the plunger 3612 presses against the ambient fluid or gas within the first lumen 3615 of the housing 3611, forcing fluid or gas into and/or against the expandable dissector 3620 and causing the expandable dissector 3620 to be moved out of the housing 3611. In the illustrated embodiment, the plunger 3612 has a volume configured to hold enough fluid (e.g., liquid or gas) to partially or completely deploy the expandable dissector 3620 when depressed. In other embodiments, depression of the plunger 3612 can cause partial evacuation of the expandable dissector 3620 or full evacuation while also partially or completely filling the expandable dissector. In further embodiments, the dissector device 3610 can exclude the plunger 3612 and the expandable dissector 3620 can instead be evacuated by depressing the handle 3651 of the syringe 3650.

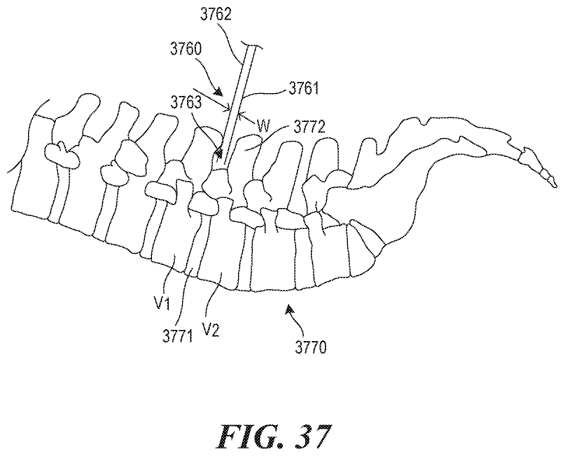

FIG. 37 is a schematic cross-sectional side view of a dilator system 3760 of the spinal surgical system 3600 of FIG. 36A in accordance with at least some embodiments of the present technology. In the illustrated embodiment, the patient's spine 3770 includes an upper vertebrae V₁ and a lower vertebrae V₂ separated by an intervertebral disc 3771. Each of the upper and lower vertebrae V₁ and V₂ includes a spinous process 3772. The dilator system 3760 includes a first dilator 3761 and a second dilator 3762. The first dilator 3761 and the second dilator 3762 can be inserted through separate ports spaced apart on the patient's skin and can be placed at different angles inside of the patient. In other embodiments, the first dilator 3761 and the second dilator 3762 can be inserted through a single port. In some embodiments, the first dilator 3761 and the second dilator 3762 can be inserted such that the distal ends of the first dilator 3761 and the second dilator 3762 meet at a position at or adjacent to the patient's spine 3770. The first dilator 3761 and the second dilator 3762 are configured to dilate the access paths to a width W. In the illustrated embodiment, the working space 3763 can be created by enlarging an existing space (e.g., a gap, lumen, hole) in the tissue by removing tissue.

In the illustrated embodiment, the working space 3763 is shown between vertebrae V₁, V₂, however, in other embodiments, the working space 3763 can saddle a spinous process 3772 or extend from one spinous process 3772 to the other spinous process 3772. The expandable dissector 3620 can be configured to enlarge the working space 3763 to a desired shape.

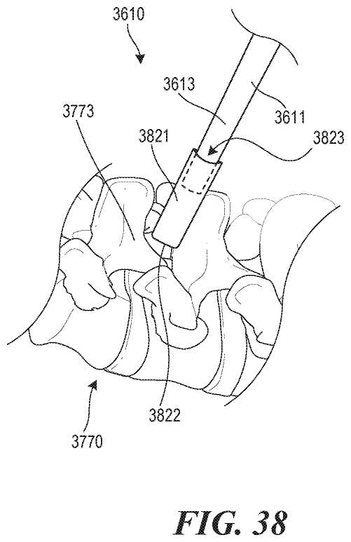

FIG. 38 is an enlarged isometric view of the dissector device 3610 of the spinal surgical system 3600 of FIG. 36A in accordance with at least some embodiments of the present technology. As aforementioned, the dissector device 3610 can include the housing 3611 and can further include a cover tip 3821. The cover tip 3821 includes a third lumen 3823 and is slidably coupled to the first proximal portion 3613 of the housing 3611. More specifically, the first proximal portion 3613 of the housing 3611 extends through the third lumen 3823. In some embodiments, the cover tip 3821 includes a spring or other biasing member configured to keep the cover tip 3821 in an expanded position (e.g., the first proximal portion 3613 of the housing 3611 is only partially within the third lumen 3823). The cover tip 3821 includes a cutting edge 3822 configured to cut soft tissues (e.g., the fascial layer near vertebrae 3773) through abrasion. In other embodiments, the cutting edge 3822 can be a blade or protrusion to aid in cutting.

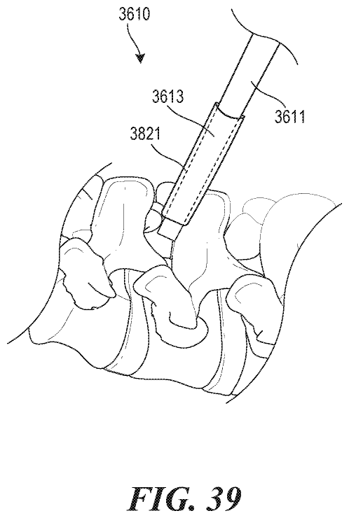

FIG. 39 is an enlarged isometric view of the dissector device 3610 of the spinal surgical system 3600 of FIG. 36A in accordance with at least some embodiments of the present technology. In the illustrated embodiment, the cover tip 3821 is retracted over the first proximal portion 3613 of the housing 3611. When the cover tip 3821 is retracted, the expandable dissector 3620 can be evacuated from the housing 3611. In some embodiments, the expandable dissector 3620 is folded between the cover tip 3821 and the housing 3611 so that it is deployed when the cover tip 3821 is retracted. In other embodiments, the expandable dissector 3620 is coupled to and/or housed in the cover tip 3821 such that the expandable dissector 3620 deploys when the cover tip 3821 is retracted. In further embodiments, the housing 3611 includes a locking mechanism configured to permanently or releasably lock the cover tip 3821 in the retracted position to ensure that the cover tip 3821 does not return to the expanded position once the expandable dissector has been deployed and/or once the cover tip 3821 has been retracted.

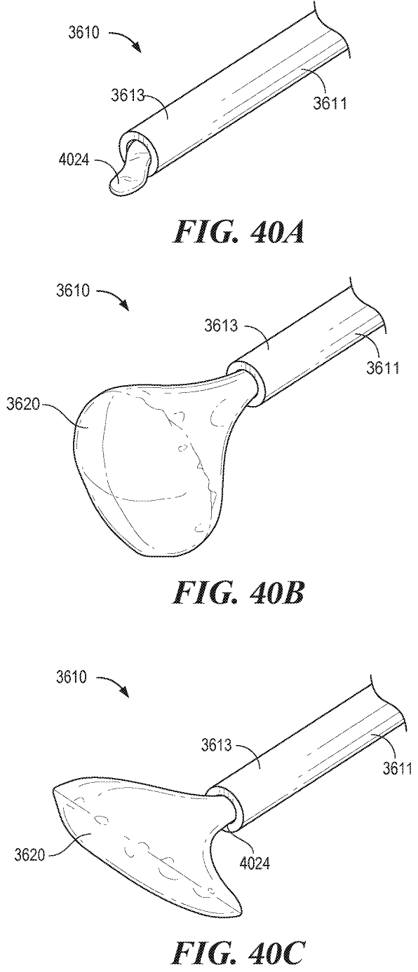

FIGS. 40A, 40B, and 40C are enlarged, isometric views of the dissector device 3610 of the spinal surgical system 3600 of FIG. 36A being deployed in accordance with at least some embodiments of the present technology. With reference to FIGS. 40A, 40B, and 40C, the housing 3611 of the dissector device 3610 includes a flange 4024 extending from the proximal portion 3631 of the housing 3611. The flange 4024 can be rounded to inhibit, limit, or prevent atraumatic injury of (e.g., cutting, bruising, traumatizing) soft tissues. The flange 4024 can be used to position, orient, and/or support the deployed expandable dissector 3620. For example, the flange 4024 can help maintain the position and/or angle of the expandable dissector 3620 relative to the housing 3611. In some embodiments, for example in embodiments without a cover tip 3821, the flange 4024 can be an alternative cutting edge. The cutting edge can be configured to cut soft tissues (e.g., the fascial layer) through abrasion.

With reference to FIG. 40A, the dissector device 3610 (shown without an optional cover tip) is in the delivery configuration, as described in detail above with reference to FIG. 36A. With reference to FIG. 40B, the dissector device 3610 is in the partially deployed configuration, as described in detail above with reference to FIG. 36B. Further, the expandable dissector 3620 has been inflated to its predetermined size and shape by depressing the handle 3651 of the syringe 3650 of FIG. 36B and/or by depressing the plunger 3612. With reference to FIG. 40C, the expandable dissector 3620 is in the fully inflated configuration, as described in detail above with reference to FIG. 36B. Further, the expandable dissector 3620 is deflated by withdrawing the handle 3651 of the syringe 3650 of FIG. 36B or by uncoupling the syringe 3650 from the stopcock 3640 and opening the stopcock 3640. In some embodiments, deflating the expandable dissector 3620 also retracts the expandable dissector 3620 into the housing 3611.

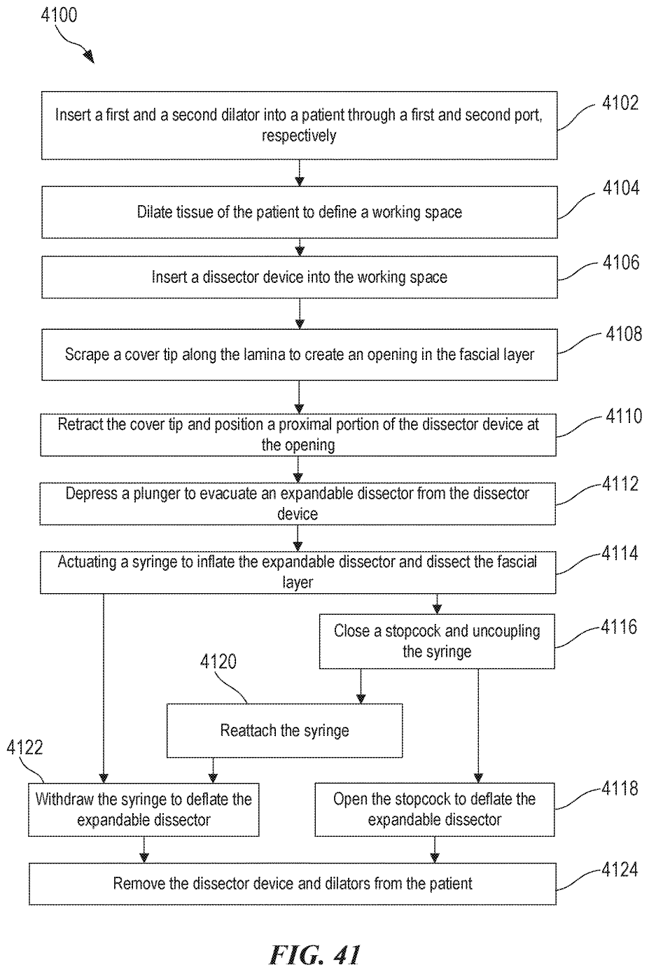

FIG. 41 is a flowchart illustrating a method 4100 for dissecting the fascial layer around a spine during a surgical procedure in accordance with at least some embodiments of the present technology.

While the method 4100 is described below with reference to the embodiments illustrated in FIGS. 36A-40C, the method 4100 can be performed using other embodiments of the present technology. Also, while the method 4100 is described below in a particular order, one or more steps can be performed in a different order or omitted entirely. The method 4100 can also include additional or alternative steps.

At block 4102, the method 4100 begins by inserting a first dilator (e.g., the first dilator 3761 of FIG. 37) and a second dilator (e.g., the second dilator 3762 of FIG. 37) into the patient through a first port and second port, respectively. The first port and/or second port can comprise a surgical incision in the patient and/or a medical port positioned therein.

The method 4100 continues at block 4104 by using the first dilator and the second dilator to dilate the tissue inside of the patient between two vertebrae of the spine and/or the tissue surrounding the fins of the vertebrae to define a working space (e.g., the working space 3763 of FIG. 37) where additional devices can be inserted and used. In some embodiments, the first dilator and the second dilator are removed after they have dilated the tissue, and the working space is retained using other mechanisms.

The method 4100 continues at block 4106 by inserting a dissector device (e.g., the dissector device 3610 of FIGS. 36A-40C) into the working space between the first and second dilators. The dissector device can be positioned in the patient via an additional port or one of the ports of the dilators. The dissector device should be able to move freely within the working space while the first and second dilators remain in a steady position relative to the working space. The method 4100 continues at block 4108 by using the cutting edge of a cover tip (e.g., the cover tip 3821 of FIGS. 38 and 39) to scrape along the tissue surrounding the lamina (e.g., the fascial layer). This creates an opening that enables the dissector device to be inserted beneath or inside the fascial layer. In some embodiments, at block 4108 a flange (e.g., the flange 4024) can be used as a cutting edge to scrape along the tissue surrounding the lamina. The method 4100 continues at block 4110 by retracting the cover tip to protect the tissue from the cutting edge of the cover tip and positioning the dissector device into or adjacent to the opening that was created at block 4108.

The method 4100 continues at block 4112 by depressing a plunger (e.g., the plunger 3612 of FIGS. 36A and 36B) to evacuate the expandable dissector (e.g., the expandable dissector 3620 of FIGS. 36A, 36B, 40B, and 40C) from a housing (e.g., the housing 3611 of FIGS. 36A and 36B) of the dissector device. In some embodiments, the expandable dissector can be evacuated by depressing a syringe (e.g., the syringe 3650 of FIGS. 36A and 36B) and the plunger can be excluded.

The method 4100 continues at block 4114 by depressing the syringe to inflate the expandable dissector with fluid or gas. As the expandable dissector expands inside the fascial layer, it dissects the fascial layer. More specifically, it separates muscle and ligament from the bone of the spine as it expands. In some embodiments, the method 4100 can further include additional operations performed by other surgical equipment while the expandable dissector is expanded. In such embodiments, the expandable dissector can act as a tamponade to reduce bleeding from the dissected fascial layer by applying constant pressure to the soft tissues.

The method 4100 can continue to either block 4116 or 4122 based on the specific surgical procedure and/or user preference. At block 4116, the user closes a stopcock (e.g., the stopcock 3640 of FIGS. 36A and 36B) to fluidly disconnect the dissector device from the syringe. After the stopcock has been closed, the syringe can be removed from the system (e.g., the spinal surgical system 3600 of FIG. 36A). The method 4100 can continue from block 4116 to either block 4118 or block 4120 based on the surgical procedure and/or user preference. At block 4118, the stopcock is opened to deflate the expandable dissector. Alternatively, at block 4120, the user can reattach the syringe to the spinal surgical system 3600. In some embodiments, between block 4116 and block 4120, the user can withdraw the syringe to fill the syringe with additional fluid or gas. Then, at block 4120, the user can use the additional fluid or gas to further inflate the expandable dissector. The method 4100 can continue from block 4120 to block 4122. At block 4122, the user can withdraw the syringe to deflate the expandable dissector after the dissection has occurred. In some embodiments, deflating the expandable dissector can also retract the expandable dissector into the housing. In further embodiments, the deflated expandable dissector can remain in place while additional operations are performed. The method 4100 continues from block 4118 or block 4122 to block 4124 by removing the dissector device, the first dilator, and the second dilator from the patient. In some embodiments, the method 4100 further comprises inserting a surgical instrument (e.g., an endoscope) in the working space defined by the first dilator and the second dilator.

### VI. Examples

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner.

1. A spinal surgical system for use in a spine procedure on a patient, the spinal surgical system comprising:
a needle guide;
a spinal needle;
a needle depth gauge;
an angle plate guide;
a scope port scalpel;
a working port scalpel;
a tissue dissector; and
a flag guide.

2. The spinal surgical system of any one of the examples herein, wherein the needle depth gauge includes (i) a slot sized to receive the spinal needle and (ii) a plurality of depth indicators configured to abut against the spinal needle and thereby indicate an insertion depth of the spinal needle in the patient.

3. The spinal surgical system of any one of the examples herein, wherein the angle plate guide includes a first base portion, a second base portion, a pivot, and a latch, wherein and the angle plate guide is transitionable between (i) an open state in which the first and second base portions are coupled together at the pivot and (ii) a closed state in which the first and second base portions are coupled together at both the pivot and the latch.

4. The spinal surgical system of any one of the examples herein, wherein the angle plate guide includes an aperture sized to receive the spinal needle.

5. The spinal surgical system of any one of the examples herein, wherein the angle plate guide includes (i) a first guiding ramp configured to guide the scope port scalpel and (ii) a second guiding ramp configured to guide the working port scalpel, wherein the first and second guiding ramps are oriented at an angle of about 30 degrees relative to one another.

6. The spinal surgical system of any one of the examples herein, wherein the angle plate guide includes (i) a first guiding ramp having a first generally V-shaped cross-section corresponding to first V-shaped sidewalls of the scope port scalpel and (ii) a second guiding ramp having a second generally V-shaped cross-section corresponding to second V-shaped sidewalls of the working port scalpel.

7. The spinal surgical system of any one of the examples herein, further comprising a guide rod, wherein the angle plate guide includes a guiding ramp having a recess sized to receive the guide rod, and wherein the working port scalpel includes a sleeve having a channel sized to receive the guide rod.

8. The spinal surgical system of any one of the examples herein, wherein the angle plate guide includes a guiding ramp having a gap sized to fit a blade of the tissue dissector therethrough.

9. The spinal surgical system of any one of the examples herein, wherein:
the angle plate guide includes (i) a first guiding ramp configured to guide the scope port scalpel and (ii) a second guiding ramp configured to guide the working port scalpel,
the first guiding ramp and the second guiding ramp are oriented at a first angle relative to one another,
the flag guide includes (i) a needle channel sized to receive the spinal needle, (ii) a first instrument channel sized to receive a visualization instrument, and (iii) a second instrument channel sized to receive a working instrument, and
the first instrument channel and the second instrument channel are oriented at the first angle relative to one another.

10. The spinal surgical system of any one of the examples herein, further comprising a plurality of angle plate guides including the angle plate guide, wherein each of the plurality of angle plate guides includes (i) a first guiding ramp configured to guide the scope port scalpel and (ii) a second guiding ramp configured to guide the working port scalpel, and wherein different ones of the angle plate guides have their respective first and second guiding ramps spaced apart by different distances.

11. The spinal surgical system of any one of the examples herein, further comprising a plurality of flag guides including the flag guide, wherein each of the plurality of flag guides includes (i) a first instrument channel sized to receive a visualization instrument, and (ii) a second instrument channel sized to receive a working instrument, and wherein different ones of the flag guides have their respective first and second instrument channels spaced apart by different distances.

12. A method for performing a multi-portal spine procedure on a patient, the method comprising:
inserting a spinal needle into the patient until the spinal needle contacts a spine of the patient;
moving a needle depth gauge relative to the spinal needle to obtain an indication of an insertion depth of the spinal needle;
selecting an angle plate guide among a plurality of angle plate guides based on the obtained indication; and
placing the selected angle plate guide on the patient and secured around the spinal needle.

13. The method of any one of the examples herein, wherein moving the needle depth gauge comprises receiving the spinal needle in a slot of the needle depth gauge and sliding the needle depth gauge until the spinal needle (i) abuts against one of a plurality of depth indicators of the needle depth gauge or (ii) is positioned above a tallest one of the plurality of depth indicators.

14. The method of any one of the examples herein, wherein each of the plurality of angle plate guides includes first and second guiding ramps, and wherein different ones of the plurality of angle plate guides have their respective first and second guiding ramps spaced apart by different distances.

15. The method of any one of the examples herein, wherein securing the selected angle plate guide comprises transitioning the selected angle plate guide from an open state to a closed state.

16. The method of any one of the examples herein, further comprising:
sliding a scope port scalpel along a first guiding ramp of the selected angle plate guide; and
sliding a working port scalpel along a second guiding ramp of the selected angle plate guide.

17. The method of any one of the examples herein, wherein sliding the working port scalpel comprises sliding a sleeve of the working port scalpel along a guide rod coupled to the selected angle plate guide.

18. The method of any one of the examples herein, further comprising maneuvering a tissue dissector along a guiding ramp of the selected angle plate guide, wherein the guiding ramp includes a gap sized to fit a blade of the tissue dissector therein.

19. The method of any one of the examples herein, further comprising:
forming a first access path and a second access path in the patient via a first scalpel and a second scalpel, respectively, each guided by the selected angle plate guide;
inserting a visualization instrument in the first access path; and
inserting a working instrument in the second access path.

20. The method of any one of the examples herein, further comprising moving a flag guide across the patient until (i) a needle channel of the flag guide receives the spinal needle, (ii) a first instrument channel of the flag guide receives the visualization instrument, and (iii) a second instrument channel of the flag guide receives the working instrument.

21. An endoscope holder for use in a surgical procedure, the endoscope holder comprising:
a handle comprising:
   a stabilizer foot configured to receive at least a portion of an endoscope, and
   a cam assembly comprising:
      a cam body defining a slot, wherein the slot is alignable with the stabilizer foot, wherein the cam body further defines a first recess and a second recess, wherein rotation of the cam assembly causes profiles of the first recess and the second recess to be aligned with the stabilizer foot, and
      a trigger coupled to the cam body and configured to rotate the cam assembly when actuated; and
a plurality of stabilizer legs removably coupled to the handle and configured to stabilize the endoscope holder when the plurality of stabilizer legs rest on a patient.

22. The endoscope holder of any one of the examples herein, wherein the profiles of the first recess and the second recess are semicircular.

23. The endoscope holder of any one of the examples herein, wherein the stabilizer foot and the cam assembly are located at a proximal portion of the handle and wherein the plurality of stabilizer legs are located at a distal portion of the handle.

24. The endoscope holder of any one of the examples herein, wherein the stabilizer foot is semicircular and wherein a semicircular recess is at a center of the stabilizer foot.

25. The endoscope holder of any one of the examples herein, wherein the plurality of stabilizer legs are removably coupled to the handle via a threaded post.

26. The endoscope holder of any one of the examples herein, wherein the plurality of stabilizer legs include one or more holes.

27. The endoscope holder of any one of the examples herein, wherein the cam assembly further comprises a biasing member configured to bias the cam assembly to a first position.

28. The endoscope holder of any one of the examples herein, wherein in the first position the slot is concentrically aligned with the stabilizer foot.

29. The endoscope holder of any one of the examples herein, wherein actuating the trigger moves the endoscope holder to a second position and resists a biasing force of the biasing member.

30. The endoscope holder of any one of the examples herein, wherein in the second position, the profiles of the first recess and the second recess are aligned with the stabilizer foot.

31. The endoscope holder of any one of the examples herein, wherein the plurality of stabilizer legs include complimentary recessed portions configured to nest with each other.

32. The endoscope holder of any one of the examples herein, wherein the handle is ergonomically designed for a user to grip the handle with their hand.

33. An endoscope holder comprising:
a stabilizer foot configured to receive at least a portion of an endoscope; and
a cam assembly comprising:
   a cam body spaced apart from the stabilizer foot and defining a slot, wherein the slot is alignable with the stabilizer foot, and
   a trigger coupled to the cam body and configured to rotate the cam assembly when actuated.

34. The endoscope holder of any one of the examples herein, further comprising a plurality of removable stabilizer legs configured to stabilize the endoscope holder.

35. The endoscope holder of any one of the examples herein, wherein the stabilizer foot and portions of the plurality of removable stabilizer legs extend in a same direction.

36. The endoscope holder of any one of the examples herein, wherein the trigger is configured to be easily actuated by a forefinger of a user when the user is gripping the endoscope holder.

37. A method of holding an endoscope with an endoscope holder comprising:
inserting the endoscope into semicircular recesses of the endoscope holder;
positioning the endoscope at a first position relative to the endoscope holder; and rotating a cam assembly of the endoscope holder via a trigger to lock the endoscope into the endoscope holder.

38. The method of any one of the examples herein, further comprising:
releasing the trigger to move the endoscope relative to the endoscope holder, and
actuating the trigger to lock the endoscope into the endoscope holder in a second position.

39. The method of any one of the examples herein, wherein rotating the cam assembly of the endoscope holder also locks the cam assembly into a rotated position.

40. The method of any one of the examples herein, further comprising coupling a plurality of stabilizer legs to a handle of the endoscope holder to provide stability to the endoscope holder.

41. A spinal surgical system for dissecting tissue during a surgical procedure, the spinal surgical system comprising:
an endoscopic dissector device including:
an inflatable dissector configured to be expanded at a working site along a spine of a patient, and
an inserter assembly configured to contain the inflatable dissector for insertion through a port in the patient, wherein the inserter assembly is operable to deliver the inflatable dissector to the working site while the inserter assembly is positioned in the port; and
an inflator assembly fluidly coupled to the endoscopic dissector device and operable to inflate the inflatable dissector located at the working site in the patient.

42. The spinal surgical system of any one of the examples herein, wherein the inserter assembly including:
a housing with a chamber configured to hold the inflatable dissector; and
a plunger configured to move the inflatable dissector out of the housing.

43. A spinal surgical system for dissecting tissue during a surgical procedure, the spinal surgical system comprising:
a dissector device comprising:
   a housing comprising a first proximal portion, a first distal portion, and a first lumen extending therethrough;
   a plunger comprising a second proximal portion, a second distal portion, and a second lumen extending therethrough, wherein the second proximal portion extends through the first lumen; and
   an expandable dissector housed within the first lumen, sealingly coupled to the housing, and having a predetermined size and shape specific to the surgical procedure, wherein the plunger is configured to evacuate the expandable dissector when depressed; and
an inflator assembly fluidly coupled to the expandable dissector via the first lumen and the second lumen, wherein the inflator assembly expands the expandable dissector when depressed.

44. The spinal surgical system of any one of the examples herein, further comprising a stopcock fluidly coupled to the inflator assembly and the dissector device and configured to control fluid flow between the inflator assembly and the expandable dissector.

45. The spinal surgical system of any one of the examples herein, wherein the housing further comprises a first stopper, wherein the plunger further comprises a second stopper, and wherein the first stopper and the second stopper are configured to control a depth of insertion of the plunger into the housing.

46. The spinal surgical system of any one of the examples herein, wherein the expandable dissector is spherical.

47. The spinal surgical system of any one of the examples herein, wherein the expandable dissector is deflated when it is evacuated from the housing.

48. The spinal surgical system of any one of the examples herein, wherein the expandable dissector is made of latex or silicon.

49. The spinal surgical system of any one of the examples herein, wherein the dissector device further comprises a retractable cover tip slidably coupled to the housing.

50. The spinal surgical system of any one of the examples herein, wherein the retractable cover tip comprises a cutting edge configured to create an opening in tissue through abrasion.

51. The spinal surgical system of any one of the examples herein, wherein retraction of the retractable cover tip evacuates the expandable dissector from the housing.

52. The spinal surgical system of any one of the examples herein, wherein the housing further comprises a flange located at the first proximal portion configured to support the expandable dissector and maintain its position relative to the dissector device.

53. The spinal surgical system of any one of the examples herein, wherein edges of the dissector device are rounded to prevent accidental trauma to non-target tissues.

54. A dissector system, comprising:
a housing comprising a first proximal portion, a first distal portion, and a first lumen extending therethrough;
a plunger comprising a second proximal portion, a second distal portion, and a second lumen extending therethrough, wherein the second proximal portion extends through a portion of the first lumen; and
an expandable dissector having a predetermined size and shape specific to a surgical procedure, wherein the plunger is configured to evacuate the expandable dissector when depressed and wherein inflating the expandable dissector dissects tissue of a patient.

55. The dissector system of any one of the examples herein, further comprising stoppers configured to inhibit movement of the plunger within the first lumen.

56. The dissector system of any one of the examples herein, wherein the dissector system is fluidly coupled to a pressure source configured to inflate the expandable dissector.

57. The dissector system of any one of the examples herein, further comprising a retractable cover tip comprising a cutting edge configured to cut soft tissue.

58. A method for dissecting tissue during a surgical procedure on a patient, the method comprising:
inserting a dissector device through a port into a working space in the patient;
using a cutting edge of the dissector device to create an opening for an expandable dissector;
positioning the dissector device adjacent to the opening;
delivering expandable dissector of the dissector device into the opening;
inflating the expandable dissector inside of the opening to dissect surrounding tissue; and
deflating the expandable dissector and removing the dissector device from the patient via the port.

59. The method of any one of the examples herein, wherein evacuating the expandable dissector and inflating the expandable dissector comprises fully depressing a syringe fluidly coupled to the expandable dissector.

60. The method of any one of the examples herein, wherein deflating the expandable dissector comprises withdrawing a syringe fluidly coupled to the expandable dissector.

61. The method of any one of the examples herein, wherein deflating the expandable dissector comprises opening a stopcock fluidly coupled to the expandable dissector.

62. The method of any one of the examples herein, wherein additional surgical operations are completed while the expandable dissector is inflated.

63. The method of any one of the examples herein, wherein evacuating the expandable dissector comprises depressing a plunger of the dissector device.

61. A spinal surgical system for use in a multi-portal spine procedure on a patient, the spinal surgical system comprising:
a spinal needle having a handle and a needle coupled to the handle, wherein the needle is configured to be inserted into the patient until the needle reaches a target site;
a plate guide selector configured to be positioned on the patient and including a first plate guide indicator and a second plate guide indicator, wherein the plate guide selector is positionable relative to the spinal needle, wherein positioning the first plate guide indicator adjacent to the spinal needle indicates a first depth in the patient, and wherein positioning the second plate guide indicator adjacent to the spinal needle indicates a second depth in the patient;
a first angle plate guide corresponding to the first plate guide indicator, wherein the first angle plate guide has a first pair of guiding ramps angled such that a first pair of imaginary lines extending along the first pair of guiding ramps intersect inside the patient substantially at the first depth; and
a second angle plate guide corresponding to the second guide indicator, wherein the second angle plate guide has a second pair of guiding ramps angled such that a second pair of imaginary lines extending along the second pair of guiding ramps intersect inside the patient substantially at the second depth,
wherein each of the first angle plate guide and the second angle plate guide is configured to facilitate triangulation of surgical instruments and/or visualization instruments toward the target site.

62. The spinal surgical system of any one of the examples herein, wherein the plate guide selector further includes a third plate guide indicator corresponding to a third depth and a fourth plate guide selector corresponding to a fourth depth, and wherein the spinal surgical system further includes:
a third angle plate guide having a third pair of guiding ramps angled such that a third pair of imaginary lines extending along the third pair of guiding ramps intersect inside the patient substantially at the third depth; and
a fourth angle plate guide having a fourth pair of guiding ramps angled such that a fourth pair of imaginary lines extending along the fourth pair of guiding ramps intersect inside the patient substantially at the fourth depth.

63. The spinal surgical system of example 62, wherein:
the first depth is between 30-45 millimeters,
the second depth is between 46-60 millimeters,
the third depth is between 61-80 millimeters, and
the fourth depth is between 81-100 millimeters.

64. The spinal surgical system of any one of the examples herein, wherein the plate guide selector further includes a slot sized to receive the needle of the spinal needle, and wherein the plate guide selector is configured to be moved laterally over the patient such that the slot moves around the needle of the spinal needle.

65. The spinal surgical system of any one of the examples herein, wherein the plate guide selector further includes a base, wherein the first plate guide indicator is spaced apart from the base by a first height, and wherein the second plate guide indicator is spaced apart from the base by a second height different than the first height.

66. The spinal surgical system of any one of the examples herein, wherein the first angle plate guide includes a first base portion, a second base portion, a pivot, and a latch, wherein the first angle plate guide is transitionable between (i) an open state in which the first and second base portions are coupled together at the pivot and (ii) a closed state in which the first and second base portions are coupled together at both the pivot and the latch.

67. The spinal surgical system of any one of the examples herein, wherein the first angle plate guide includes an aperture sized to receive the needle of the spinal needle.

68. The spinal surgical system of any one of the examples herein, further comprising a scope port scalpel and a working port scalpel, wherein a first one of the first pair of guiding ramps is configured to guide the scope port scalpel, wherein a second one of the first pair of guiding ramps is configured to guide the working port scalpel, and wherein the first and second ones of the first pair of guiding ramps are oriented at an angle of about 30 degrees relative to one another.

69. The spinal surgical system of any one of the examples herein, further comprising a scope port scalpel and a working port scalpel, wherein a first one of the first pair of guiding ramps includes a first generally V-shaped cross-section corresponding to V-shaped sidewalls of the scope port scalpel, and wherein a second one of the first pair of guiding ramps includes a second generally V-shaped cross-section corresponding to V-shaped sidewalls of the working port scalpel.

70. The spinal surgical system of any one of the examples herein, further comprising a guide rod and a working port scalpel, wherein a first one of the first pair of guiding ramps includes a recess sized to receive the guide rod, and wherein the working port scalpel includes a sleeve having a channel sized to receive the guide rod therethrough.

71. The spinal surgical system of any one of the examples herein, further comprising a flag guide including (i) a needle channel sized to receive the needle of the spinal needle, (ii) a first instrument channel sized to receive a visualization instrument, and (iii) a second instrument channel sized to receive a working instrument, wherein the first instrument channel and the second instrument channel are oriented relative to one another at an angle equal to an angle at which the first pair of guiding ramps are oriented relative to one another.

72. The spinal surgical system of any one of the examples herein, wherein the first pair of guiding ramps are separated from one another by a first distance, and wherein the second pair of guiding ramps are separated from one another by a second distance different from the first distance.

73. The spinal surgical system of any one of the examples herein, further comprising a plurality of flag guides, wherein each of the plurality of flag guides includes (i) a first instrument channel sized to receive a visualization instrument, and (ii) a second instrument channel sized to receive a working instrument, and wherein different ones of the flag guides have their respective first and second instrument channels spaced apart by different distances.

### VII. Conclusion

It will be apparent to those having skill in the art that changes may be made to the details of the above-described embodiments without departing from the underlying principles of the present disclosure. In some cases, well known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Additional details regarding systems, devices, and methods for performing spine procedures and, in particular, multi-portal spine procedures are provided in U.S. Pat. Nos. 11,464,648, 11,678,906, 10,105,238, U.S. Patent Application Publication No. 2023/0320866, U.S. Patent Application Publication No. 2025/0082478, U.S. Patent Application Publication No. 2024/0009003, U.S. Patent Application Publication No. 2024/0299187, U.S. Patent Application Publication No. 2024/0325006, U.S. Patent Application Publication No. 2024/0390031, U.S. patent application Ser. No. 18/988,467, U.S. patent application Ser. No. 18/987,830, U.S. Pat. Nos. 9,820,788, 10,799,367, PCT Application No. PCT/US25/14790, U.S. Pat. No. 10,322,009, the disclosures of which are incorporated by reference herein in their entireties. To the extent any material incorporated herein by reference conflicts with the present disclosure, the present disclosure controls.

Where the context permits, singular or plural terms may also include the plural or singular term, respectively. For example, throughout this disclosure, the singular terms “a,” “an,” and “the” include plural referents unless the context clearly indicates otherwise. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, a step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

Reference herein to “one embodiment,” “an embodiment,” “some embodiments” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. The terms “about,” “approximately,” and “substantially” as used herein shall be interpreted to mean within ±10% of the stated value. Additionally, all ranges disclosed herein are to be understood to encompass the endpoints, and any and all subranges subsumed therein. For example, a range of “1 to 10” includes any and all subranges between (and including) the minimum value of 1 and the maximum value of 10 (e.g., any and all subranges having a minimum value of equal to or greater than 1 and a maximum value of equal to or less than 10, such as 5.5 to 10).

The disclosure set forth above is not to be interpreted as reflecting an intention that any claim or example requires more features than those expressly recited in that claim or example. Rather, as the preceding examples and the following claims reflect, inventive aspects lie in a combination of fewer than all features of any single foregoing disclosed embodiment. Thus, the preceding examples and the following claims are hereby expressly incorporated into the Detailed Description, with each claim standing on its own as a separate embodiment. This disclosure includes all permutations of the independent claims with their dependent claims.

What is claimed is:

1. A spinal surgical system for use in a multi-portal spine procedure on a patient, the spinal surgical system comprising:
a spinal needle having a handle and a needle coupled to the handle, wherein the needle is configured to be inserted into the patient until the needle reaches a target site;
a plate guide selector configured to be positioned on the patient and including a first plate guide indicator and a second plate guide indicator, wherein the plate guide selector is positionable relative to the spinal needle, wherein positioning the first plate guide indicator adjacent to the spinal needle indicates a first depth in the patient, and wherein positioning the second plate guide indicator adjacent to the spinal needle indicates a second depth in the patient;
a first angle plate guide corresponding to the first plate guide indicator, wherein the first angle plate guide has a first pair of guiding ramps angled such that a first pair of imaginary lines extending along the first pair of guiding ramps intersect inside the patient substantially at the first depth; and
a second angle plate guide corresponding to the second guide indicator, wherein the second angle plate guide has a second pair of guiding ramps angled such that a second pair of imaginary lines extending along the second pair of guiding ramps intersect inside the patient substantially at the second depth,
wherein each of the first angle plate guide and the second angle plate guide is configured to facilitate triangulation of surgical instruments and/or visualization instruments toward the target site.

2. The spinal surgical system of claim 1, wherein the plate guide selector further includes a third plate guide indicator corresponding to a third depth and a fourth plate guide selector corresponding to a fourth depth, and wherein the spinal surgical system further includes:
a third angle plate guide having a third pair of guiding ramps angled such that a third pair of imaginary lines extending along the third pair of guiding ramps intersect inside the patient substantially at the third depth; and
a fourth angle plate guide having a fourth pair of guiding ramps angled such that a fourth pair of imaginary lines extending along the fourth pair of guiding ramps intersect inside the patient substantially at the fourth depth.

3. The spinal surgical system of claim 2, wherein:
the first depth is between 30-45 millimeters,
the second depth is between 46-60 millimeters,
the third depth is between 61-80 millimeters, and
the fourth depth is between 81-100 millimeters.

4. The spinal surgical system of claim 1, wherein the plate guide selector further includes a slot sized to receive the needle of the spinal needle, and wherein the plate guide selector is configured to be moved laterally over the patient such that the slot moves around the needle of the spinal needle.

5. The spinal surgical system of claim 1, wherein the plate guide selector further includes a base, wherein the first plate guide indicator is spaced apart from the base by a first height, and wherein the second plate guide indicator is spaced apart from the base by a second height different than the first height.

6. The spinal surgical system of claim 1, wherein the first angle plate guide includes a first base portion, a second base portion, a pivot, and a latch, wherein the first angle plate guide is transitionable between (i) an open state in which the first and second base portions are coupled together at the pivot and (ii) a closed state in which the first and second base portions are coupled together at both the pivot and the latch.

7. The spinal surgical system of claim 1, wherein the first angle plate guide includes an aperture sized to receive the needle of the spinal needle.

8. The spinal surgical system of claim 1, further comprising a scope port scalpel and a working port scalpel, wherein a first one of the first pair of guiding ramps is configured to guide the scope port scalpel, wherein a second one of the first pair of guiding ramps is configured to guide the working port scalpel, and wherein the first and second ones of the first pair of guiding ramps are oriented at an angle of about 30 degrees relative to one another.

9. The spinal surgical system of claim 1, further comprising a scope port scalpel and a working port scalpel, wherein a first one of the first pair of guiding ramps includes a first generally V-shaped cross-section corresponding to V-shaped sidewalls of the scope port scalpel, and wherein a second one of the first pair of guiding ramps includes a second generally V-shaped cross-section corresponding to V-shaped sidewalls of the working port scalpel.

10. The spinal surgical system of claim 1, further comprising a guide rod and a working port scalpel, wherein a first one of the first pair of guiding ramps includes a recess sized to receive the guide rod, and wherein the working port scalpel includes a sleeve having a channel sized to receive the guide rod therethrough.

11. The spinal surgical system of claim 1, further comprising a flag guide including (i) a needle channel sized to receive the needle of the spinal needle, (ii) a first instrument channel sized to receive a visualization instrument, and (iii) a second instrument channel sized to receive a working instrument, wherein the first instrument channel and the second instrument channel are oriented relative to one another at an angle equal to an angle at which the first pair of guiding ramps are oriented relative to one another.

12. The spinal surgical system of claim 1, wherein the first pair of guiding ramps are separated from one another by a first distance, and wherein the second pair of guiding ramps are separated from one another by a second distance different from the first distance.

13. The spinal surgical system of claim 1, further comprising a plurality of flag guides, wherein each of the plurality of flag guides includes (i) a first instrument channel sized to receive a visualization instrument, and (ii) a second instrument channel sized to receive a working instrument, and wherein different ones of the flag guides have their respective first and second instrument channels spaced apart by different distances.

14. A method for performing a multi-portal spine procedure on a patient, the method comprising:
inserting a spinal needle into the patient until the spinal needle contacts a spine of the patient;
moving a needle depth gauge relative to the spinal needle to obtain an indication of an insertion depth of the spinal needle;
selecting an angle plate guide among a plurality of angle plate guides based on the obtained indication; and
placing the selected angle plate guide on the patient and secured around the spinal needle.

15. The method of claim 14, wherein moving the needle depth gauge comprises receiving the spinal needle in a slot of the needle depth gauge and sliding the needle depth gauge until the spinal needle (i) abuts against one of a plurality of depth indicators of the needle depth gauge or (ii) is positioned above a tallest one of the plurality of depth indicators.

16. The method of claim 14, wherein each of the plurality of angle plate guides includes first and second guiding ramps, and wherein different ones of the plurality of angle plate guides have their respective first and second guiding ramps spaced apart by different distances.

17. The method of claim 14, wherein placing the selected angle plate guide comprises transitioning the selected angle plate guide from an open state to a closed state.

18. The method of claim 14, further comprising:
sliding a scope port scalpel along a first guiding ramp of the selected angle plate guide; and
sliding a working port scalpel along a second guiding ramp of the selected angle plate guide.

19. The method of claim 18, wherein sliding the working port scalpel comprises sliding a sleeve of the working port scalpel along a guide rod coupled to the selected angle plate guide.

20. The method of claim 14, further comprising maneuvering a tissue dissector along a guiding ramp of the selected angle plate guide, wherein the guiding ramp includes a gap sized to fit a blade of the tissue dissector therein.

21. The method of claim 14, further comprising:
forming a first access path and a second access path in the patient via a first scalpel and a second scalpel, respectively, each guided by the selected angle plate guide;
inserting a visualization instrument in the first access path; and
inserting a working instrument in the second access path.

22. The method of claim 21, further comprising moving a flag guide across the patient until (i) a needle channel of the flag guide receives the spinal needle, (ii) a first instrument channel of the flag guide receives the visualization instrument, and (iii) a second instrument channel of the flag guide receives the working instrument.
